(19) United States
(12) Patent Application Publication
(10) Pub. No.: US 2021/0000911 A1
(43) Pub. Date: Jan. 7, 2021

(54) COMPOSITIONS AND METHODS FOR PROTECTING EPITHELIAL AND BARRIER INTEGRITY

(71) Applicants: OCUNEXUS THERAPEUTICS, INC., San Diego, CA (US); AUCKLAND UNISERVICES LIMITED, Auckland (NZ)

(72) Inventors: Odunayo Omolola Boluwarin MUGISHO, Auckland (NZ); Colin Richard GREEN, Auckland (NZ); Bradford James DUFT, Solana Beach, CA (US)

(21) Appl. No.: 16/856,784

(22) Filed: Apr. 23, 2020

Related U.S. Application Data
(60) Provisional application No. 62/837,697, filed on Apr. 23, 2019.

(51) Int. Cl. A61K 38/10; A61K 31/353; A61K 9/00

(52) U.S. Cl. CPC A61K 38/10; A61K 9/0053; A61K 9/0019; A61K 31/353

(57) ABSTRACT

The inventions relate to the use of hemichannel blockers to modulate BRB integrity and function, RPE integrity and function, tight junction integrity and function, ZO-1 internalization, gap junction internalization, and/or type IV collagen levels in a subject.

### RELATED PATENT APPLICATIONS

This application claims benefit to U.S. Provisional Application No. 62/837,697, filed Apr. 23, 2019, and is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 10, 2020, is named E3697-00571_SL.txt and is 58,718 bytes in size.

### FIELD

The inventions relate generally to the retina, and particularly to the retinal pigment epithelium and the blood retinal barrier.

### INCORPORATION BY REFERENCE

All U.S. patents, U.S. patent application publications, foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content was repeated in the application, and will be treated as part of the text and content of this application as filed.

### BACKGROUND

The following includes information that may be useful in understanding the present inventions. It is not an admission that any of the information, publications or documents specifically or implicitly referenced herein is prior art, or essential, to the presently described or claimed inventions.

Connexins are proteins that form gap junctions, intercellular channels that connect the cytoplasm of two neighbouring cells and allow the movement of ions, metabolites and signalling molecules between the cells following the docking of two gap junction half-channels, called hemichannels. Various connexin isotypes are expressed in the human body with connexin43 being the most common. Studies have shown that connexin43 channels contribute to the processes of inflammation, cell migration and physiological roles such as the coordination of cardiac myocyte contraction, amongst other things.

Connexin channels are expressed in virtually all tissues of the body, except for mature skeletal muscle and mobile cell types such as sperm and erythrocytes. One gap junction is composed of two connexons (or hemichannels), which connect across the intercellular space between adjacent cells and allow intracellular molecules to flow between those cells. Each connexon of a gap junction resides in the adjacent cell membrane and is formed by the covalent oligomerization of six individual connexin (“Cx”) proteins. The prerequisite for the formation of functional gap junctions is the assembly of connexin proteins into hemichannels and their insertion into the membrane. For intercellular communication hemichannels from one cell must dock to their counterparts on the opposing membrane of an adjacent cell to allow the transmission of molecules via gap junctions from one cell to the other.

Connexin43, a ubiquitously expressed, 43 kDa protein, has also been linked, however, to a number of pathological conditions with several studies providing evidence that undocked connexin43 hemichannels, rather than gap junction channels themselves, facilitate various connexin43-mediated deleterious processes. These include ionic imbalances and the onset of calcium waves, an inflow of cytotoxic molecules from the extracellular space into cells, and ATP release through open hemichannels triggering the inflammasome pathway. Inflammasomes are multimeric protein complexes that assemble upon sensing of a variety of stress factors. Their formation results in caspase-1-mediated activation and secretion of the pro-inflammatory cytokines pro-interleukin (IL)-1β and IL-18, which induce an inflammatory response.

Some reports indicate that connexin hemichannels open primarily only during pathological situations such as ischemic or hypoxic stress. Recent research supports the idea that connexin43 hemichannel opening may contribute to lesion spread after injury such as retinal ischemia and spinal cord injury. See Chen Y S, et al. (2015) Neuroprotection in the treatment of glaucoma—A focus on connexin43 gap junction channel blockers. Eur J Pharm Biopharm 95 (Pt B):182-193; Danesh-Meyer H V, et al. (2012) Connexin43 mimetic peptide reduces vascular leak and retinal ganglion cell death following retinal ischaemia. Brain 135 (Pt 2):506-520; Guo C X, et al. (2016) Connexin43 Mimetic Peptide Improves Retinal Function and Reduces Inflammation in a Light-Damaged Albino Rat Model. Invest Ophthalmol Vis Sci 57 (10):3961-3973; Kerr N M, et al. (2012) High pressure-induced retinal ischaemia reperfusion causes upregulation of gap junction protein connexin43 prior to retinal ganglion cell loss. Exp Neurol 234 (1):144-152; Zhang J, et al. (2013) Connexin based therapeutic approaches to inflammation in the central nervous system. In: Connexin Cell Communication Channels: Roles in the Immune System and Immunopathology. Taylor and Francis Group, CRC Press, Boca Raton, Fla., pp 273-305.

In the retina, connexin43 is expressed by vascular endothelial, glial and retinal pigment epithelial (RPE) cells. These are cell types that make up the inner and outer blood-retinal barrier (BRB). The outer BRB, in particular, plays a crucial role in the retina as it separates the highly vascularised choroid, which provides 80% of the retinal blood supply, from the rest of the retina. RPE cells are also physiologically important and regulate retinal glucose homeostasis, angiogenic balance and photoreceptor functioning. RPE cell pathology has been implicated in many retinal diseases including diabetic retinopathy, a chronic retinal disease that occurs due to hyperglycaemia-linked vascular pathology characterised in its late stages by BRB leakage and neovascularisation with formation of new and leaky blood vessels. Durham J T, Herman I M (2011) Microvascular modifications in diabetic retinopathy. Curr Diab Rep 11 (4):253-264.

Studies have shown that pathological events characteristic of diabetic retinopathy occur primarily in the presence of both hyperglycaemia and inflammation, and that blocking connexin43 hemichannels can protect against pro-inflammatory cytokine release and NLRP3 inflammasome activation in RPE cells. See Mugisho O O, et al. (2018) The inflammasome pathway is amplified and perpetuated in an autocrine manner through connexin43 hemichannel mediated ATP release. Biochim Biophys Acta 1862 (3):385-393.

Notwithstanding these findings, however, whether and how connexin43 may impact the barrier properties of RPE cells remains unknown. This is particularly important because loss of RPE-mediated BRB integrity is a key feature of diabetic macular edema, a consequence of diabetic retinopathy that induces vision loss.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

The pigmented layer of the retina, or retinal pigment epithelium (RPE) is the pigmented cell layer just outside the neurosensory retina that nourishes retinal visual cells and is firmly attached to the underlying choroid and overlying retinal visual cells. Increases in the permeability of the retinal pigment epithelium and blood retinal barrier are involved in a number of diseases, disorders and conditions.

This patent describes the use of hemichannel blockers to attenuate disruption of retinal pigment epithelium and blood retinal barrier integrity.

The patent also describes the use of hemichannel blockers to attenuate ZO-1 internalization.

The patent also describes the use of hemichannel blockers to attenuate connexin, particularly connexin 43, internalization.

The patent also describes the use of hemichannel blockers to attenuate collagen IV upregulation.

The inventions relate, in one aspect, for example, to the use of hemichannel blockers to modulate RPE permeability in a subject, including in conditions characterized in whole or in part by loss of RPE integrity.

In another aspect, provided are methods for modulating BRB integrity in a subject.

In another aspect, provided are methods for modulating tight junction integrity in a subject.

In another aspect, provided are methods for modulating type IV collagen in a subject.

In another aspect, methods are provided for confirming, measuring or evaluating the activity of compounds useful for modulating RPE permeability, BRB permeability, ZO-1 internalization, collagen IV regulation, and/or connexin hemichannel internalization using assays described herein. Assays include tests using ARPE-19 cells. See Dunn K C, et al., ARPE-19, a human retinal pigment epithelial cell line with differentiated properties. Exp Eye Res. 1996 February; 62(2):155-69. In one embodiment, the test assay is an ARPE-19 cell RPE breakdown assay using trans-epithelial resistance (TEER) and FITC-dextran dye leak across an ARPE-19 monolayer, for example, to measure RPE layer permeability in the presence of known or potential hemichannel blockers.

This patent describes, in part, the use of compounds and methods to modulate connexin hemichannels, including connexin 43 hemichannels, to block or modulate RPE permeability and improve or maintain RPE integrity.

This patent also describes, in part, the use of compounds and methods to modulate connexin hemichannels, including connexin 43 hemichannels, to block or modulate BRB permeability and improve or maintain BRB integrity.

Methods of the invention will be useful in attenuating abnormal, elevated, dysregulated and/or otherwise undesired levels of RPE permeability in a subject by administration of a connexin hemichannel blocker to a subject who would benefit therefrom. Methods of the invention will be also useful in attenuating abnormal, elevated, dysregulated and/or otherwise undesired levels of BRB permeability in a subject by administration of a connexin hemichannel blocker to a subject who would benefit therefrom.

Methods of the invention will be useful in attenuating abnormal, elevated, dysregulated and/or otherwise undesired levels of collagen IV in a subject by administration of a connexin hemichannel blocker to a subject who would benefit therefrom. For example, type VI collagen formation is associated with higher arterial stiffness in people with type 1 diabetes, and can be treated with the compounds and compositions of the invention. Type 1 diabetes have increased risk of cardiovascular disease. Large artery stiffness is an important determinant of cardiovascular risk, and arterial stiffness, and has been shown to be a strong predictor of mortality and cardiovascular outcome. Arterial stiffening reflects fragmentation and loss of elastin fibers and accumulation of collagen fibers in the media of large arteries. Until now, however, the mechanisms responsible for arterial stiffening remain incompletely understood. They are based at least in part on hemichannel opening in face of high glucose and baseline inflammation, and arterial stiffness, by way of example, may be treated with modulators of hemichannel opening to lessen collagen IV as described herein.

Methods of the invention will be also useful in attenuating abnormal, elevated, dysregulated and/or otherwise undesired levels of ZO-1 and/or tight junction disruption in a subject by administration of a connexin hemichannel blocker to a subject who would benefit therefrom. Methods of the invention will be also useful in attenuating abnormal, elevated, dysregulated and/or otherwise undesired levels of connexin hemichannel internalization in a subject by administration of a connexin hemichannel blocker to a subject who would benefit therefrom. These include, for example, subjects with inflammatory bowel diseases and inflammatory bowel disease associated colorectal cancer, which are characterized by inflammation that compromises the integrity of the epithelial barrier, and where apical tight junction proteins are critical in the maintenance of epithelial barrier function and control of paracellular permeability.

It is an object of the invention to provide methods for attenuating abnormal, elevated, dysregulated and/or otherwise undesired levels of RPE permeability, BRB permeability, tight junction breakdown, ZO-1 internalization, connexin internalization, or type IV collagen production in a subject by administration of a connexin hemichannel blocker to a subject who would benefit therefrom.

It is another object of the invention to provide compounds, compositions, formulations, kits and methods for the treatment of diseases, disorders and conditions that will benefit from modulation of RPE permeability to maintain or enhance RPE integrity.

It is another object of the invention to provide compounds, compositions, formulations, kits and methods for the treatment of diseases, disorders and conditions that will benefit from modulation of BRB permeability to maintain or enhance BRB integrity. Objects of the invention also include providing compounds, compositions, formulations, kits and methods for the treatment of diseases, disorders and conditions that will benefit from modulation of tight junction breakdown, ZO-1 internalization, connexin internalization, and/or type IV collagen production.

In some aspects, the method of treatment is applied to mammals, e.g., humans.

In another aspect, the invention provides a hemichannel blocker for the treatment of one or more diseases, disorders and conditions as described herein.

Hemichannel blockers useful in the present invention include compounds of Formula I, for example Xiflam, and/or an analogue or prodrug of any of the foregoing compounds, or a peptidomimetic, such as Peptagon (aka Peptide5) or an analogue or prodrug thereof, or another hemichannel blocker, and other hemichannel blocker compounds described or incorporated by reference herein.

Some preferred hemichannel blockers include small molecule hemichannel blockers (e.g., Xiflam (tonabersat)). In some embodiments, the hemichannel blocker is a small molecule other than Xiflam, for example, a hemichannel blocker described in Formula I or Formula II in US Pat. App. Publication No. 20160177298, filed in the name of Colin Green, et al., the disclosure of which is hereby incorporated in its entirety by this reference, as noted above. Various preferred embodiments include use of a small molecule that blocks or ameliorates or otherwise antagonizes or inhibits hemichannel opening, to treat diseases, disorders and conditions characterized at least in part by abnormal, elevated, dysregulated and/or otherwise undesired, unwanted or detrimental levels of RPE or BRB or tight junction integrity, including those described or referenced herein, as well as the treatment of diseases, disorders and conditions that will benefit from modulation of RPE or BRB or tight junction integrity, tight junction breakdown, ZO-1 internalization, connexin internalization, and/or type IV collagen production. In various embodiments, the small molecule that blocks or ameliorates or inhibits hemichannel opening is a prodrug of Xiflam or an analogue thereof.

In other embodiments, hemichannel blockers include peptide and peptidomimetic hemichannel blockers (e.g., Peptagon, VDCFLSRPTEKT (SEQ ID NO: 1), a peptidomimetic), and other peptidomimetic hemichannel blockers comprising or consisting essentially of or consisting of the amino acids sequence SRPTEKT (SEQ ID NO: 2), as well as other peptide hemichannel modulating agents, including, for example, Gap 19, etc. In another embodiment, the hemichannel blocker is Peptide5, GAPS, GAP19, GAP26, GAP27 or α-connexin carboxy-terminal (ACT) peptides, e.g., ACT-1 or other active anti-hemichannel peptidomimetic. In any of the aspects of this invention, the hemichannel blockers are connexin peptides or peptidomimetics, including peptides or peptidomimetics comprising, consisting essentially of, or consisting of connexin extracellular domains, transmembrane regions, and connexin carboxy-terminal peptides. The connexin hemichannel blocking peptides or peptidomimetics may be modified or unmodified. The connexin hemichannel blocking peptides or peptidomimetics are made chemically, synthetically, or otherwise manufactured. In some embodiments, the connexin hemichannel blocking peptides or peptidomimetics are Cx43 peptides or peptidomimetics. In some aspects, the therapeutically effective modified or unmodified peptide or peptidomimetic comprises a portion of an extracellular or transmembrane domain of a connexin, such as Cx43 or Cx45, for example, a portion of a connexin Extracellular Loop 2, including a portion of Cx43 Extracellular Loop 2 and a portion of Cx45 Extracellular Loop 2.

In another aspect, the invention provides the use of a hemichannel blocker in the manufacture of a medicament for use in the treatment of one or more diseases, disorders and conditions described or referred to herein. The medicament will comprise, consist essentially of, or consist of a hemichannel blocker. In one embodiment, the medicament will comprise, consist essentially of, or consist of a peptide hemichannel blocker. In one embodiment, the medicament will comprise, consist essentially of, or consist of a peptidomimetic hemichannel blocker. In one embodiment, the medicament will comprise, consist essentially of, or consist of a small molecule hemichannel blocker. In one embodiment, the medicament will comprise, consist essentially of, or consist of a compound according to Formula I or Formula II in US Pat. App. Publication No. 20160177298. In one embodiment, the medicament will comprise, consist essentially of, or consist of Xiflam (tonabersat).

The term “comprising,” which is synonymous with “including,” “containing,” or “characterized by,” is inclusive or open-ended and does not exclude additional, unrecited elements or ingredients from the medicament (or steps, in the case of a method). The phrase “consisting of” excludes any element, step, or ingredient not specified in the medicament (or steps, in the case of a method). The phrase “consisting essentially of” refers to the specified materials and those that do not materially affect the basic and novel characteristics of the medicament (or steps, in the case of a method). The basic and novel characteristics of the inventions are described throughout the specification, and include the ability of medicaments and methods of the invention to block or modulate connexin gap junction hemichannels and to modulate one or more of BRB integrity, RPE integrity, tight junction integrity, BRB breakdown, RPE breakdown, tight junction breakdown, ZO-1 internalization, connexin internalization, and/or type IV collagen production, as the case may be. Material changes in the basic and novel characteristics of the inventions, including the medicaments and methods described herein, include an unwanted or clinically undesirable, detrimental, disadvantageous or adverse diminution of hemichannel modulation and/or modulation of one or more of BRB integrity, RPE integrity, tight junction integrity, BRB breakdown, RPE breakdown, tight junction breakdown, ZO-1 internalization, connexin internalization, and/or type IV collagen production, as the case may be BRB breakdown, RPE breakdown, tight junction breakdown, ZO-1 internalization, connexin internalization. In one embodiment, the medicament will comprise, consist essentially of, or consist of a connexin 43 hemichannel blocker, for example, a peptidometic or small molecule connexin 43 hemichannel blocker. In one preferred embodiment, the medicament will comprise or consist essentially of Xiflam (tonabersat), or another compound of Formula I.

In another aspect, the invention provides the use of a hemichannel blocker in the manufacture of a medicament (or a package or kit containing one or more medicaments and/or containers, with or without instructions for use) for modulation of a hemichannel and/or treatment of any of the diseases, disorders and/or conditions described or referred to herein. In one aspect, for example, the invention provides the use of a connexin hemichannel blocker, including, for example, Xiflam and/or an analogue thereof or Peptagon or an analogue thereof, in the manufacture of a medicament or package or kit for the treatment of a disorder where modulation of a hemichannel for a purpose described herein may be of benefit. In one embodiment, the medicament will comprise, consist essentially of, or consist of a connexin 43 hemichannel blocker, for example, a peptidometic or small molecule connexin 43 hemichannel blocker. In one embodiment, the hemichannel blocker composition useful in the invention may include a pharmaceutically acceptable carrier and may be formulated as a pill, a solution, a microsphere, a nanoparticle, an implant, a matrix, or a hydrogel formulation, for example, or may be provided in lyophilized form.

The hemichannel being modulated for the purposes described herein may be any connexin of interest for that purpose.

Within the intestine, for example, the range of connexins includes the following: connexin 26 (Cx26), connexin 32 (Cx32), connexin 36 (Cx36), connexin 37 (Cx37), connexin 43 (Cx43), and connexin 45 (Cx45).

In various embodiments, by way of example, the hemichannel being modulated comprises one or more of Cx26, connexin 30 (Cx30), Cx32, Cx37, connexin 40 (Cx40), Cx43, and Cx45. In one embodiment, the hemichannel being modulated comprises one or more of a Cx37, Cx40, or Cx43 protein. In one particular embodiment, the hemichannel and/or hemichannel being modulated comprises Cx43. In some embodiments, the hemichannel being modulated can include or exclude any of the foregoing connexins. In some aspects, the hemichannel blocker is a blocker of a Cx37 hemichannel, a Cx43 hemichannel, a Cx40 hemichannel and/or a Cx45 hemichannel. In certain preferred embodiments, the hemichannel blocker is a connexin 43 hemichannel blocker. The pharmaceutical compositions of this invention for any of the uses featured herein may also comprise a hemichannel blocker that may inhibit or block Cx26, Cx30, Cx32, Cx36, Cx37, Cx40, Cx43, Cx45, or any other connexin, or connexin hemichannel. In one embodiment the hemichannel blocker blocks a connexin hemichannel in a blood vessel. In other embodiments the hemichannel blocker blocks a connexin hemichannel in a blood microvessel. In other embodiments the hemichannel blocker blocks a connexin hemichannel in a capillary. In other embodiments the hemichannel blocker blocks a connexin hemichannel in endothelium.

Another embodiment of this aspect of the invention provides a pharmaceutical pack that includes a small molecule or other hemichannel blocker. In one embodiment, the hemichannel blocker is Xiflam. In another embodiment, the hemichannel blocker is Peptagon.

In another aspect of the invention, the effects of hemichannel blocker treatment in a subject is evaluated or monitored using methods for monitoring RPE or BRB integrity, tight junction integrity, or collagen IV production.

The activity of hemichannel blockers may be evaluated using certain biological assays. Effects of known or candidate hemichannel blockers on molecular motility can be identified, evaluated, or screened for using the methods described in the Examples below, or other art-known or equivalent methods for determining the passage of compounds through connexin hemichannels. Various methods are known in the art, including dye transfer experiments, for example, transfer of molecules labelled with a detectable marker, as well as the transmembrane passage of small fluorescent permeability tracers, which has been widely used to study the functional state of hemichannels. Various embodiments of this aspect of the invention are described herein, including a method for use in identifying or evaluating the ability of a compound to block hemichannels, which comprises: (a) bringing together a test sample and a test system, said test sample comprising one or more test compounds, and said test system comprising a system for evaluating hemichannel block, said system being characterized in that it exhibits, for example, elevated transfer of a dye or labelled metabolite, for example, in response to the introduction of hypoxia or ischemia to said system, a mediator of inflammation, or other compound or event that induces hemichannel opening, such as a drop in extracellular Ca²⁺; and, (b) determining the presence or amount of a rise in, for example, the dye or other labelled metabolite(s) in said system. Positive and/or negative controls may be used as well. Optionally, a predetermined amount of hemichannel blocker (e.g., Peptagon or Xiflam) may be added to the test system. As noted herein, in one embodiment, hemichannel blockers, such as Peptagon and Xiflam, for example, exhibit activity in an in vitro assay on the order of less than about 1 to 5 nM, preferably less than about 10 nM and more preferably less than about 50 pM. In an in vivo assay these compounds preferably show hemichannel block at a concentration of less than about 10-100 micromolar (μM), and more preferably at a concentration of less than about 50 μM. Other hemichannel blockers may be within these ranges, and also within a range of less than about 200 pM. Assay methods are provided for confirming, measuring or evaluating the activity of hemichannel modulating compounds useful as described herein. Assays include tests using ARPE-19 cells. See Dunn K C, et al., ARPE-19, a human retinal pigment epithelial cell line with differentiated properties. Exp Eye Res. 1996 February; 62(2):155-69. In one embodiment, the test assay is an ARPE-19 cell RPE breakdown assay using trans-epithelial resistance (TEER) and FITC-dextran dye leak across an ARPE-19 monolayer, for example, to measure RPE layer permeability in the presence of known or potential hemichannel blockers.

### BRIEF DESCRIPTION OF THE FIGURES

FIGS. 1A-1B show that treatment using hemichannel block with Peptide5 prevented a decrease in TEER and an increase in FITC-dextran permeability following HG and cytokines in ARPE-19 cells. FIG. 1A shows culturing cells with a combination of HG and cytokines resulted in a statistically significant decrease in TEER at 48 h (p=0.0007) and 72 h (p=0.0030). Peptide5 treatment prevented a decrease in TEER at both time-points with no statistically significant difference in TEER between Peptide5-treated and basal cells. Statistical analysis was carried out using two-way ANOVA with Dunnett's multiple comparison's test. FIG. 1B shows culturing cell with a combination of HG and cytokines also led to an increase in FITC-dextran permeability across the cell monolayer at 72 h (p=0.0016) but this was prevented with Peptide5 treatment relative to basal conditions. Statistical analysis was carried out using one-way ANOVA with Dunnett's multiple comparison's test. n=3; ** p≤0.01; *** p≤0.001

FIGS. 2A-2B show that treatment using hemichannel block Peptide5 prevented cell membrane destabilisation following HG and cytokines application. HG and cytokines did not induce cell death but led to a subtle but significance release of LDH (p<0.0001). However, Peptide5 treatment prevented LDH release relative to basal conditions. Statistical analysis was carried out using one-way ANOVA with Dunnett's multiple comparison's test. **** p<0.0001; n=3; scale-bar=100 μm.

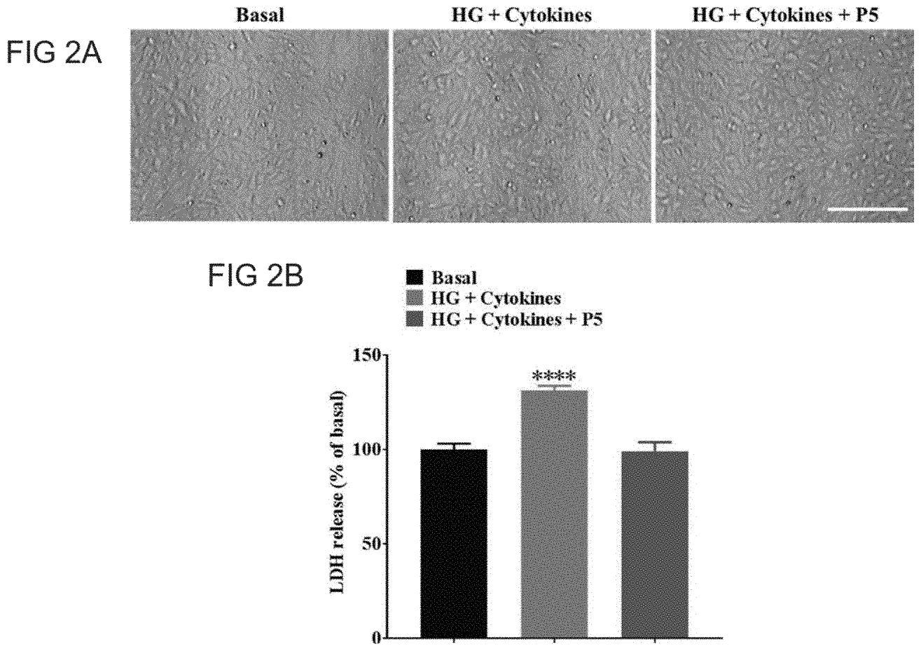

FIG. 3 shows that treatment using hemichannel block Peptide5 protected against loss of ZO-1 (green) localisation at the cell membrane. Basally, ZO-1 was localised to cell membrane (white arrows). With HG and cytokines, there was a loss of membrane localisation and an increase in internalisation of ZO-1 to the cell cytoplasm. However, Peptide5 treatment maintained ZO-1 localisation to cell membranes similar to basal conditions. Scale bar=50 μm.

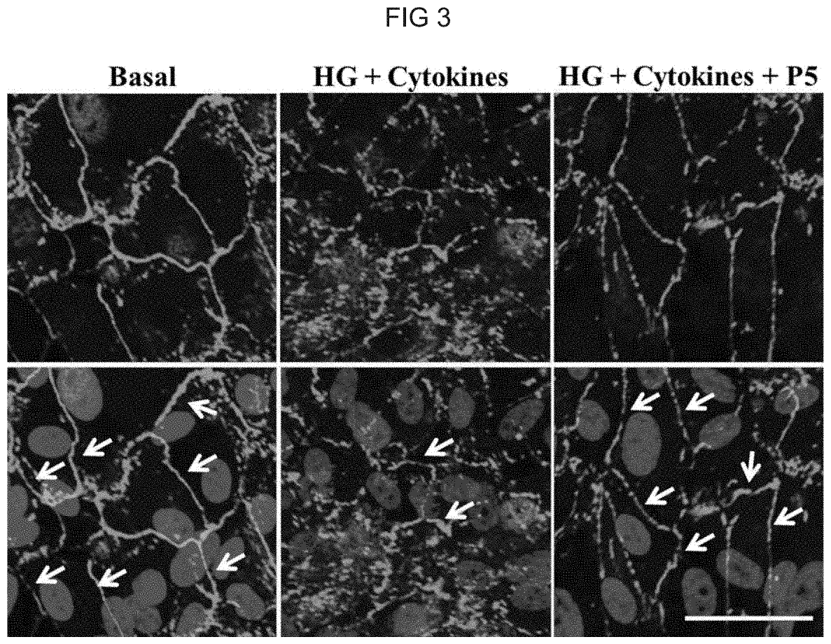

FIG. 4 shows that treatment using hemichannel block with Peptide5 prevented collagen IV (red) upregulation following HG and cytokines application. HG and cytokines resulted in upregulation of collagen IV relative to basal conditions (p=0.0180). With Peptide5 treatment, collagen IV expression was maintained at basal levels with no difference in expression between Peptide5-treated and basal cells. Scale bar=100 μm; Statistical analysis was carried out using one-way ANOVA with Dunnett's multiple comparison's test. * p<0.05; n=3.

FIGS. 5A-5C show that exogenously added ATP reverses the protection conferred by Peptide5 following application of HG and cytokines. FIG. 5A shows HG and cytokines increased ATP release relative to basal conditions (p=0.0014) while Peptide5 treatment was able to reduce ATP release (p=0.0003) so that there was no difference between basal and Peptide5 treatment conditions. Statistical analysis was carried out using one-way ANOVA with Dunnett's multiple comparison's test. ** p≤0.01; *** p≤0.001; n.s.=not significant; n=3. FIG. 5B shows LDH release induced by HG and cytokines was reduced by Peptide5 treatment (p<0.0001). However, in the presence of ATP, LDH levels were increased to injury levels with no significant difference between HG and cytokine-injured and HG, cytokines, Peptide5, and ATP-injured cells. Statistical analysis was carried out using one-way ANOVA with Dunnett's multiple comparison's test. **** p<0.0001; n.s.=not significant; n=3. FIG. 5C shows Peptide5 treatment protected against the redistribution of connexin43 protein (green) from cell membrane plaques. Basally, connexin43 protein was localised to cell membranes (white arrows). With HG and cytokines, there was a loss of membrane plaque localisation and an increase in internalisation of connexin43 to the cells. However, Peptide5 treatment maintained connexin43 primarily in gap junction plaques, similar to basal conditions. In the presence of exogenously added ATP, there was again a loss of connexin43 at cell membranes, similar to the HG and cytokines group. Scale bar=50 μm.

### DETAILED DESCRIPTION

The retinal pigment epithelium (RPE) is a specialized epithelium lying in the interface between the neural retina and the choriocapillaris where it forms the outer blood-retinal barrier (BRB). The main functions of the RPE are the following: (1) transport of nutrients, ions, and water, (2) absorption of light and protection against photooxidation, (3) reisomerization of all-trans-retinal into 11-cis-retinal, which is crucial for the visual cycle, (4) phagocytosis of shed photoreceptor membranes, and (5) secretion of essential factors for the structural integrity of the retina.

In the healthy eye, the RPE secretes pigment epithelium-derived factor (PEDF), which helps to maintain the retinal as well as the choriocapillaris structure in two ways, both as a neuroprotective factor and as an antiangiogenic factor that can inhibit endothelial cell proliferation and stabilized the endothelium of the choriocapillaris. Reviewed by Simo R, et al., The Retinal Pigment Epithelium: Something More than a Constituent of the Blood-Retinal Barrier—Implications for the Pathogenesis of Diabetic Retinopathy Journal of Biomedicine and Biotechnology Volume 2010, Article ID 190724, 15 pages. Another vasoactive factor synthesized by the RPE is vascular endothelial growth factor (VEGF), which is secreted in low concentrations by the RPE in the healthy eye where it prevents endothelial cell apoptosis, is essential for an intact endothelium of the choriocapillaris, and also acts as a permeability factor stabilizing the fenestrations of the endothelium. Id. In a healthy eye, PEDF and VEGF are secreted at opposite sides of the RPE. PEDF is secreted to the apical side where it acts on neurons and photoreceptors whereas most of VEGF is secreted to the basal side where it acts on the choroidal endothelium. Id. Overproduction of VEGF has been described in the development of proliferative diabetic retinopathy and diabetic macular edema, and downregulation of PEDF expression by elevated glucose concentration in cultured human RPE cells has been observed, leading to the proposal for strategies in blocking VEGF or stimulating PEDF as new therapeutic approaches for diabetic retinopathy.

This application relates to the surprising discovery of the modulation of hemichannel opening has direct and immediate effects on the maintenance and enhancement of RPE and BRB integrity. See Examples 1-6 below. It has been surprisingly discovered that connexin hemichannels can mediate and play a key role in BRB integrity and RPE integrity, discoveries that have important implications in the treatment of various diseases, disorders and conditions characterized in whole or in part by loss of BRB and/or RPE integrity and, importantly, their increased permeability.

It has also been discovered that hemichannel blockers including, for example, connexin 43 hemichannel blockers, can be used to attenuate ZO-1 internalization. Thus, such hemichannel blockers can be used for methods to modulate barrier permeability for preventing barrier dysfunction in disease states.

It has also been discovered that hemichannel blockers including, for example, connexin 43 hemichannel blockers, can be used to attenuate type IV collagen upregulation. Thus, hemichannel blockers can be used for methods to modulate upregulation of collagen IV in disease states.

The inventors have shown that high glucose (HG) and cytokine application results in a decrease in trans-epithelial resistance (TEER) and an increase in FITC-dextran dye leak across a monolayer of RPE cells. Furthermore, results showed that this loss of RPE barrier integrity was not due to cell death but instead was caused by internalisation of the tight junction protein, ZO-1, and led to upregulation of collagen IV.

Importantly, as shown in Examples 1-6, connexin43 hemichannel block was found to protect against a decrease in TEER, an increase in FITC-dextran dye leak, the internalisation of ZO-1 and the up-regulation in collagen IV deposition. Studies have reported increases in collagen IV secretion in human DME patients compared to controls. Mugisho O O, et al. (2018) Intravitreal pro-inflammatory cytokines in non-obese diabetic mice: Modelling signs of diabetic retinopathy. PLoS One 13 (8):e0202156.

To better understand the mechanism by which hemichannels mediate these processes, ATP was restored into the culture medium in the presence of the Peptide5 hemichannel blocker. Results showed that ATP reversed the protection conferred by hemichannel block such that there was no difference between cells with HG and cytokines alone and cells with HG, cytokines, Peptide5 and ATP in terms of LDH release and localisation of connexin43 gap junction plaques.

### Definitions

A “small molecule” is defined herein to have a molecular weight below about 600 to 900 daltons, and is generally an organic compound. A small molecule can be an active agent of a hemichannel blocker prodrug. In one embodiment, the small molecule is below 600 daltons. In another embodiment, the small molecule is below 900 daltons.

As used herein, “treatment” (and grammatical variations thereof such as “treat” or “treating”) refers to clinical intervention to alter the natural course of the individual, tissue or cell being treated, and can be performed either for prophylaxis or during clinical pathology. Desirable effects of treatment include, but are not limited to, preventing occurrence or recurrence of a disease, disorder or condition, alleviation of signs or symptoms, diminishment of any direct or indirect pathological consequences of the disease, decreasing the rate of disease progression, amelioration or palliation of the disease state, and remission or improved prognosis. In some embodiments, compounds, methods and compositions of the invention can be used to delay development of a disease, disorder or condition, or to slow the progression of a disease, disorder or condition. The term does not necessarily imply that a subject is treated until total recovery. Accordingly, “treatment” includes reducing, alleviating or ameliorating the symptoms or severity of a particular disease, disorder or condition or preventing or otherwise reducing the risk of developing a particular disease, disorder or condition. It may also include maintaining or promoting a complete or partial state of remission of a condition. “Treatment” as used herein also includes improving RPE integrity, BRB integrity, and tight junction integrity in a subject, and/or lowering collagen IV production in a subject, following administration of a hemichannel blocker.

The term “treating” a disease, condition or disorders or the like, may refer to preventing, slowing, reducing, decreasing, stopping and/or reversing the disorder, disease or condition, and/or maintain or improving RPE or BRB integrity, tight junction integrity, attenuating RPE or BRB or tight junction breakdown, ZO-1 internalization, connexin internalization, and/or collagen IV production.

The term “preventing” means preventing in whole or in part, or ameliorating or controlling.

As used herein, “effective amount” refers to an amount effective, at dosages and for periods of time necessary, to achieve the desired therapeutic or prophylactic result. For example, and not by way of limitation, an “effective amount” can refer to an amount of a compound or composition, disclosed herein, that is able to treat the signs and/or symptoms of a disease, disorder or condition that involve impaired BRB integrity, impaired RPE integrity, impaired tight junction integrity, or increased collagen IV production, and so on, as described herein, or to an amount of a hemichannel compound or composition that is able to beneficially modulate impaired BRB integrity, impaired RPE integrity, impaired tight junction integrity, and/or increased collagen IV production.

As used herein, “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is preferably also one in which any toxic or detrimental effects of the substance/molecule, agonist or antagonist may be outweighed by the therapeutically beneficial effects. A therapeutically effective amount of a hemichannel blocker will beneficially modulate impaired BRB integrity, impaired RPE integrity, impaired tight junction integrity, and/or increased collagen IV production in a subject.

As used herein, “prophylactically effective amount” refers to an amount effective, at dosages and for periods of time necessary, to achieve a desired prophylactic result. Typically, but not necessarily, since a prophylactic dose is used in subjects prior to or at an earlier stage of a disease, disorder or condition, the prophylactically effective amount will be less than the therapeutically effective amount.

The term “pharmaceutical formulation” refers to a preparation which is in such form as to permit the biological activity of an active ingredient contained therein, e.g., a hemichannel blocker, to be effective, and which does not contain additional components that are unacceptably toxic to a subject to whom the formulation would be administered.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, buffers, excipients, stabilizers, and preservatives.

As used herein, the term “subject” or the like, including “individual,” and “patient”, all of which may be used interchangeably herein, refers to any mammal, including humans, domestic and farm animals, and zoo, wild animal park, sports, or pet animals, such as dogs, horses, cats, sheep, pigs, cows, etc. The preferred mammal is a human, including adults, children, and the elderly. Preferred sports animals are horses and dogs. Preferred pet animals are dogs and cats. The subject may be, for example, an aquatic park animal, such as a dolphin, whale, seal or walrus. In certain embodiments, the subject, individual or patient is a human.

As used herein, the term “hemichannel” is a part of a gap junction (two hemichannels or connexons connect across an intercellular space between adjacent cells to form a gap junction) and is comprised of a number of connexin proteins, typically homologous or heterologous, i.e., homo- or hetero-meric hexamers of connexin proteins, that form the pore for a gap junction between the cytoplasm of two adjacent cells. The hemichannel is supplied by a cell on one side of the junction, with two hemichannels from opposing cells normally coming together to form the complete intercellular hemichannel. However, in some cells, and in cells under some circumstances, the hemichannel itself is active as a conduit between the cytoplasm and the extracellular space allowing the transfer of ions and small molecules.

Compounds of Formula I, for example Xiflam, and/or an analogue or pro-drug of any of the foregoing compounds, can modulate the function and/or activity of hemichannels, preferably those comprising any type of connexin protein. Accordingly, reference to “hemichannel” should be taken broadly to include a hemichannel comprising, consisting essentially of, or consisting of any one or more of a number of different connexin proteins, unless the context requires otherwise. However, by way of example, a hemichannel may comprise one or more of any connexin, including those referred to specifically above. In one embodiment, a hemichannel consists of one of the aforementioned connexins. In one embodiment, a hemichannel comprises one or more of connexin 26, 30, 32, 36, 37, 40, 45 and 47. In one embodiment, a hemichannel consists of one of connexin 37, 40, or 43. In one embodiment, the hemichannel is a connexin 43 hemichannel. In one embodiment, a hemichannel is a vascular hemichannel. In one embodiment, a hemichannel is a connexin hemichannel found in vascular endothelial cells. In one embodiment, a hemichannel is a connexin hemichannel found in vascular smooth muscle cells. In one embodiment, a hemichannel is a connexin hemichannel found in endothelial or epithelial cells outside the vasculature (for example, intestinal endothelium or epithelium). In one particular embodiment, a hemichannel comprises one or more of connexin 30, 37 and connexin 43. In one particular embodiment, a hemichannel consists of connexin 30. In one particular embodiment, a hemichannel consists of connexin 37. In one particular embodiment, a hemichannel consists of connexin 43. In one embodiment, the hemichannel comprises one or more connexin excluding connexin 26. In one embodiment, the composition can include or exclude a hemichannel blocker of any connexin, including the foregoing.

Hemichannels and hemichannels may be present in cells of any type. Accordingly, reference to a “hemichannel” or a “hemichannel” should be taken to include reference to a hemichannel or hemichannel present in any cell type, unless the context requires otherwise. In one embodiment of the invention, the hemichannel or hemichannel is present in a cell in an organ, or in a cancer or tumor. In one embodiment, the hemichannel is a vascular hemichannel. In one embodiment, the hemichannel is a connexin hemichannel found in vascular endothelial cells and/or vascular smooth muscle cells.

As used herein, “modulation of a hemichannel” is the modulation of one or more functions and/or activities of a hemichannel, typically, the flow of molecules between cells through a hemichannel. Such functions and activities include, for example, the flow of molecules from the extracellular space or environment through a hemichannel into a cell, and/or the flow of molecules through a hemichannel from the intracellular space or environment of a cell into the extracellular space or environment. Compounds useful for modulation of a hemichannel may be referred to as “hemichannel modulators.”

Modulation of the function of a hemichannel may occur by any means. However, by way of example only, modulation may occur by one or more of: inducing or promoting closure of a hemichannel; preventing, blocking, inhibiting or decreasing hemichannel opening; triggering, inducing or promoting cellular internalization of a hemichannel and/or gap junction. Use of the words such as “blocking”, “inhibiting”, “preventing”, “decreasing” and “antagonizing”, and the like, may not be taken to imply complete blocking, inhibition, prevention, or antagonism, although this may be preferred, and shall be taken to include partial blocking, inhibition, prevention or antagonism to at least reduce the function or activity of a hemichannel and/or hemichannel. Similarly, “inducing” or “promoting” should not be taken to imply complete internalization of a hemichannel (or group of hemichannels), and should be taken to include partial internalization to at least reduce the function or activity of a hemichannel.

As used herein, the term “hemichannel blocker” is a compound that interferes with the passage of molecules through a connexin hemichannel. A hemichannel blocker can block or decrease hemichannel opening, block or reduce the release of molecules through a hemichannel to an extracellular space, and/or block or reduce the entry of molecules through a hemichannel into an intracellular space. Hemichannel blockers include compounds that fully or partially block hemichannel leak or the passage of molecules to or from the extracellular space. Hemichannel blockers also include compounds that decrease the open probability of a hemichannel. Open probability is a measure of the percentage of time a channel remains open versus being closed (reviewed in Goldberg G S, et al., Selective permeability of gap junction channels Biochimica et Biophysica Acta 1662 (2004) 96-101). Examples of hemichannel blockers include peptides, small molecules, antibodies and antibody fragments. Hemichannel blockers include hemichannel modulators. Hemichannel blockers may interfere directly, or directly, with the passage of molecules through a connexin hemichannel.

As used herein, the terms “modulation of RPE integrity” and “modulating BRB integrity” refer to maintaining or improving integrity and/or function, or slowing a decrease in RPE or BRB integrity and/or function. It also refers to improving, i.e., lowering unwanted increases in, permeability, for example. RPE or BRB integrity modulation is accomplished with a hemichannel blocker, and is useful in the treatment of disease, disorders and conditions characterized in whole or in part by pathological, abnormal or otherwise unwanted or undesired decreases in RPE or BRB integrity and/or function. Compounds useful for modulation of RPE or BRB integrity may be referred to as “RPE or BRB modulators.” The compounds of the invention may be used in methods of treatment to modulate RPE or BRB integrity, wherein RPE or BRB integrity is modulated, e.g., where RPE or BRB integrity is improved, leveled and/or smoothed, including in methods of treatment of diseases, disorders or conditions characterized in whole or in part by pathological, abnormal or otherwise unwanted or undesired dimunition of RPE or BRB integrity. Integrity of the RPE and BRB is essential to prevent the unregulated leakage of materials across the barrier created by intercellular adhesions and tight junctions between cells.

As used herein, the terms “modulation of tight junction integrity” and “modulating tight junction integrity” refer to maintaining or improving integrity and/or function, or slowing a decrease in tight junction integrity and/or function. It also refers to improving, i.e., lowering unwanted increases in, permeability, for example. Tight junction integrity modulation is accomplished with a hemichannel blocker, and is useful in the treatment of disease, disorders and conditions characterized in whole or in part by pathological, abnormal or otherwise unwanted or undesired decreases in tight junction integrity and/or function. Compounds useful for modulation of tight junction integrity may be referred to as “tight junction modulators.” The compounds of the invention may be used in methods of treatment to modulate tight junction integrity, wherein tight junction integrity is modulated, e.g., where tight junction integrity is improved, levelled and/or smoothed, including in methods of treatment of diseases, disorders or conditions characterized in whole or in part by pathological, abnormal or otherwise unwanted or undesired dimunition of tight junction integrity.

As used herein, the terms “modulation of type IV collagen” and “modulating type IV collagen” refer to lowering or slowing an increase in type IV collagen production. It also refers to improving, i.e., lowering unwanted increases in type IV collagen production. Modulation of type IV collagen production is accomplished with a hemichannel blocker, and is useful in the treatment of disease, disorders and conditions characterized in whole or in part by pathological, abnormal or otherwise unwanted or undesired increases in type IV collagen production. Compounds useful for modulation of type IV collagen production may be referred to as “type IV collagen modulators.” The compounds of the invention may be used in methods of treatment to modulate type IV collagen production, wherein type IV collagen production is modulated, e.g., where type IV collagen production is decreased, slowed, levelled and/or smoothed, including in methods of treatment of diseases, disorders or conditions characterized in whole or in part by pathological, abnormal or otherwise unwanted or undesired increases in type IV collagen production.

The inflammasome is a multiprotein complex comprising caspase 1, PYCARD, NALP, and optionally caspase 5 (also known as caspase 11 or ICH-3). The exact composition of an inflammasome depends on the activator that initiates inflammasome assembly. Inflammasomes promote the maturation of the inflammatory cytokines interleukin 1β (IL-1β) and interleukin 18 (IL-18). Hemichannel blockers according to the invention can modulate or regulate inflammasome activity and inflammasome pathway activation. Target inflammasomes for hemichannel blockers include the NLRP3 inflammasome.

The terms “peptide,” “peptidomimetic” and “mimetic” include synthetic or genetically engineered chemical compounds that may have substantially the same structural and functional characteristics of protein regions which they mimic. In the case of connexin hemichannels, these may mimic, for example, the extracellular loops of hemichannel connexins.

As used herein, the term “peptide analogs” refer to the compounds with properties analogous to those of the template peptide and can be non-peptide drugs. “Peptidomimetics” (also known as peptide mimetics) which include peptide and peptide-based compounds, also include such non-peptide based compounds such as peptide analogs. Peptidomimetics that are structurally similar to therapeutically useful peptides can be used to produce an equivalent or enhanced therapeutic or prophylactic effect. Peptides and peptidomimetics may, in some aspects, be modified or unmodified. Generally, peptidomimetics are structural or functional mimics (e.g., identical or similar) to a paradigm polypeptide (i.e., a polypeptide that has a biological or pharmacological function or activity), but can also have one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. The mimetic can be either entirely composed of natural amino acids, synthetic chemical compounds, non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. In the case of connexin hemichannels, these can mimic, for example, hemichannel extracellular loops which are involved in connexon-connexon docking and cell-cell channel formation. Peptidomimetics encompass those described herein, as well as those as may be known in the art, whether now known or later developed. Peptides and peptimimetic hemichannel blockers may also be modified to increase stability, improve bioavailability and/or to increase cell membrane permeability.

The patent describes new methods to modulate BRB integrity and function, RPE integrity and function, tight junction integrity and function, and type IV collagen production. BRB integrity, RPE integrity, tight junction integrity, and type IV collagen production is abnormal, dysregulated, or disordered, and may be improved by the methods of the invention in a number of diseases, disorders or conditions, some of which are characterized by unwanted or pathologic levels of BRB permeability, RPE permeability, tight junction disruption, and/or type IV collagen production.

Blockers of hemichannel opening, including hemichannel blockers, include small peptide and small molecule blockers.

The instant inventions provide, inter alia, methods for modulation of BRB integrity, RPE integrity, tight junction integrity, and type IV collagen production by administration of a hemichannel blocker, such as Peptagon, and/or an analogue thereof, compounds of Formula I, for example Xiflam, and/or an analogue or pro-drug of any of the foregoing compounds, for the treatment of a disease, disorder or condition where RPE modulation, BRB modulation, tight junction modulation and/or type IV collagen modulation may be of benefit.

In some embodiments, this invention features the use of compounds of Formula I, for example Xiflam, and/or an analogue or pro-drug of any of the foregoing compounds to directly and immediately block Cx43 hemichannels and to cause a concentration and time-dependent modulation of RPE integrity, BRB integrity, tight junction integrity and/or modulation of type IV collagen production.

### Connexins

In various embodiments, the hemichannel being modulated is any connexin hemichannel. In certain embodiments, the hemichannel being modulated is a hemichannel, a connexin 26 (Cx26) hemichannel, a connexin 30 (Cx30) hemichannel, a connexin 32 (Cx32) hemichannel, a connexin 36 (Cx36) hemichannel, a connexin 37 (Cx37) hemichannel, a connexin 40 (Cx40) hemichannel, a connexin 40.1 (Cx40.1) hemichannel, a connexin 43 (Cx43) hemichannel, a connexin 45 (Cx45) hemichannel, a connexin 46 (Cx46) hemichannel, a connexin 47 (Cx47) hemichannel. In one embodiment, the hemichannel being modulated comprises one or more of a Cx26, Cx30, Cx32, Cx36, Cx37, Cx40, Cx43, Cx45 and/or Cx47 protein. In one particular embodiment, the hemichannel and/or hemichannel being modulated is a Cx37 and/or Cx40 and/or Cx43 hemichannel. In one particular embodiment, the hemichannel and/or hemichannel being modulated is a Cx30 and/or Cx43 and/or Cx45 hemichannel. In some embodiments, the hemichannel being modulated can include or exclude any of the foregoing connexin proteins. In some aspects, the hemichannel blocker is a blocker of a Cx43 hemichannel, a Cx40 hemichannel and/or a Cx45 hemichannel. In certain preferred embodiments, the hemichannel blocker is a connexin 43 blocker. The pharmaceutical compositions of this invention for any of the uses featured herein may also comprise a hemichannel blocker that may inhibit or block Cx26, Cx30, Cx31.1, Cx36, Cx37, Cx40, Cx45, Cx50, or Cx57 hemichannels, or any other connexin hemichannel (including homologous and heterologous hemichannels. In some embodiments the hemichannel being modulated can include or exclude any of the foregoing connexin hemichannels, or can be a heteromeric hemichannel.

The hemichannel blocker used in any of the administration, co-administrations, compositions, kits or methods of treatment of this invention is a Cx43 hemichannel blocker, in one embodiment. Other embodiments include Cx45 hemichannel blockers, Cx30 hemichannel blockers, Cx37 hemichannel blockers, Cx40 hemichannel blockers, and blockers of a Cx26, Cx31.1, Cx36, Cx50, and/or Cx57 hemichannel or a hemichannel comprising, consisting essentially of, or consisting of any other connexins noted above or herein. Some embodiments may include or exclude any of the foregoing connexins or hemichannels, or others noted in this patent.

### Hemichannel Blockers

### Small Molecule Hemichannel Blockers

Examples of hemichannel blockers include small molecule hemichannel blockers (e.g., Xiflam (tonabersat). In some embodiments, the hemichannel blocker is a small molecule other than Xiflam, for example, a hemichannel blocker described in Formula I. Various preferred embodiments include use of a small molecule that blocks or ameliorates or otherwise antagonizes or inhibits hemichannel opening, to treat the diseases, disorders and conditions described or referenced herein. In various embodiments, the small molecule that blocks or ameliorates or inhibits hemichannel opening is a prodrug of Xiflam or an analogue thereof.

In some embodiments, this invention features the use of small molecule hemichannel blockers including, for example, compounds of Formula I, such as Xiflam, and/or an analogue or pro-drug of any of the foregoing compounds to block Cx43 hemichannels, for example, and to cause a concentration and time-dependent modulation of RPE integrity and function, BRB integrity and function, tight junction integrity and function and/or modulation of type IV collagen production.

By way of example, the hemichannel blocker Xiflam may be known by the IUPAC name N-[(3S,4S)-6-acetyl-3-hydroxy-2,2-dimethyl-3,4-dihydrochromen-4-yl]-3-chloro-4-fluorobenzamide or (3S-cis)-N-(6-acetyl-3,4-dihydro-3-hydroxy-2,2-(dimethyl-d6)-2H-1-benzopyran-4-yl)-3-chloro-4-fluorobenzamide.

In one embodiment, Xiflam and/or an analogue or prodrug thereof is chosen from the group of compounds having the Formula I:

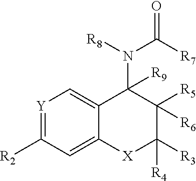

wherein,

### Y is C—R₁;

R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O—, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

For any of the Markush groups set forth above, that group can include or exclude any of the species listed for that group. Hemichannel blockers for use in methods of the invention may include or exclude any of these compounds.

In another embodiment, the analogue of Formula I is the compound carabersat (N-[(3R,4S)-6-acetyl-3-hydroxy-2,2-dimethyl-3,4-dihydrochromen-4-yl]-4-fluorobenzamide) or trans-(+)-6-acetyl-4-(S)-(4-fluorobenzoylamino)-3,4-dihydro-2,2-dimethyl-2H-1-benzo[b]pyran-3R-ol, hemihydrate.

In certain embodiments, Xiflam and/or an analogue thereof are in the form of a free base or a pharmaceutically acceptable salt. In other embodiments, one or more polymorph, one or more isomer, and/or one or more solvate of Xiflam and/or an analogue thereof may be used.

In another embodiment, a hemichannel modulating compound is chosen from the group of compounds having the Formula II:

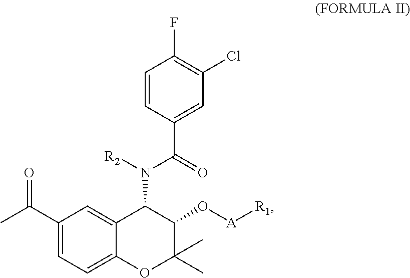

wherein
Q is O or an oxime of formula ═NHOR₄₃, wherein R₄₃ is
(i) selected from H, C₁₋₄ fluoroalkyl or optionally substituted C₁₋₄ alkyl, or
(ii) -A₃₀₀-R₃₀₀ wherein
A₃₀₀ is a direct bond, —C(O)O*—, —C(R₃)(R₄)O*—, —C(O)O—C(R₃)(R₄)O*—, or C(R₃)(R₄)OC(O)O*— wherein the atom marked* is directly connected to R₃₀₀,
R₃ and R₄ are selected independently from H, fluoro, C₁₋₄ alkyl, or C₁₋₄ fluoroalkyl, or
R₃ and R₄ together with the atom to which they are attached form a cyclopropyl group,
R₃₀₀ is selected from groups [1], [2], [2A], [3], [4], [5] or [6];
R₂ is H or B—R₂₁,

A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁, R₃ and R₄ are selected independently from H, fluoro, C₁₋₄ alkyl, or C₁₋₄ fluoroalkyl, or R₃ and R₄ together with the atom to which they are attached form a cyclopropyl group,

R₁ is selected from groups [1], [2], [2A], [3], [4], [5] and [6] wherein the atom marked ** is directly connected to A:

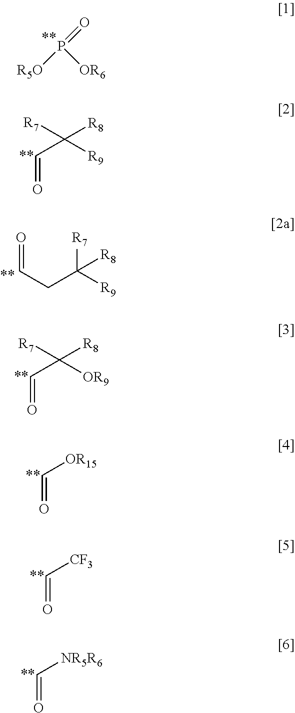

R₅ and R₆ are each independently selected from H, C₁₋₄ alkyl, C₁₋₄ fluoroalkyl, and

benzyl;

R₇ is independently selected from H, C₁₋₄ alkyl, and C₁₋₄ fluoroalkyl;

R₈ is selected from:

(i) H, C₁₋₄ alkyl, or C₁₋₄ fluoroalkyl,

(ii) the side chain of a natural or unnatural alpha-amino acid, or a peptide as described herein, and

(iii) biotin or chemically linked to biotin;

R₉ is selected from H, —N(R₁₁)(R₁₂), —N⁺(R₁₁)(R₁₂)(R₁₃)X⁻, and —N(R₁₁)C(O)R₁₄

wherein R₁₁, R₁₂, and R₁₃ are independently selected from H, C₁₋₄ alkyl, and C₁₋₄ fluoroalkyl,

R₁₄ is H, C₁₋₄ alkyl, or C₁₋₄ fluoroalkyl,

R₁₅ is selected from C₁₋₄ alkyl and C₁₋₄ fluoroalkyl,

X⁻ is a pharmaceutically acceptable anion,

wherein,

B is a direct bond, —C(O)O*—, —C(R₂₃)(R₂₄)O*, C(O)O C(R₂₃)(R₂₄)*, or

C(R₂₃)(R₂₄)OC(O)O* wherein the atom marked * is directly connected to R₂₁,

R₂₃ and R₂₄ are selected independently from H, fluoro, C₁₋₄ alkyl, and C₁₋₄ fluoroalkyl,

R₂₁ is selected from groups [21], [22], [22A], [23], [24], [25] and [26] wherein the atom marked ** is directly connected to B:

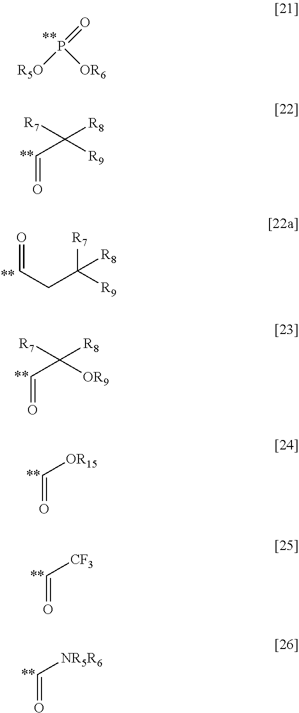

The hemichannel blockers for use in methods of the invention may include or exclude any of the compounds of Formula I of II, for example.

### Peptide and Peptidomimetic Hemichannel Blockers

In other embodiments, this invention features the use of peptide hemichannel blockers, for example, peptidomimetic compounds, such as Peptagon, block connexin hemichannels and to cause a concentration and time-dependent reduction in modulation of RPE integrity, BRB integrity, tight junction integrity and/or modulation of type IV collagen production. Hemichannel blockers may include peptides corresponding to specific sequences within extracellular loops E1 and E2 involving the conserved QPG and SHVR (SEQ ID NO: 172) motifs of E1 (Gap26 peptide) and the SRPTEK (SEQ ID NO: 173) motif in E2 (Gap27 peptide) as well as the cytoplasmic loop (Gap19 peptide). The hemichannel blockers for use in methods of the invention may include or exclude any of the “Gap” compounds. The most potent peptidomimetic is Peptagon (VDCFLSRPTEKT) (SEQ ID NO:1). Preferred peptidomimetic compounds include the SRPTEKT (SEQ ID NO: 2), 7-mer motif.

In some embodiments, peptide and/or peptidomimetic hemichannel blockers (e.g., Peptagon) comprise connexin extracellular domains, transmembrane regions, and connexin carboxy-terminal peptides. The connexin hemichannel blocking peptides or peptidomimetics may be modified or unmodified. The connexin hemichannel blocking peptides or peptidomimetics are made chemically, synthetically, or otherwise manufactured. In some embodiments, the connexin hemichannel blocking peptides or peptidomimetics are Cx43 peptides or peptidomimetics. In some aspects, the therapeutically effective modified or unmodified peptide or peptidomimetic comprises a portion of an extracellular or transmembrane domain of a connexin, such as Cx43 or Cx45, for example, a portion of a connexin Extracellular Loop 2, including a portion of Cx43 Extracellular Loop 2 and a portion of Cx45 Extracellular Loop 2. In some aspects peptide or peptidomimetic comprises a portion of an extracellular or transmembrane domain of connexin Cx26, Cx30, Cx31.1, Cx36, Cx37, Cx40, Cx50, Cx57, or another connexin mentioned herein. Peptidomimetics corresponding to a portion of Cx43 Extracellular Loop 2 are presently preferred.

Peptagon is a hemichannel blocker that can operate in a dose dependent manner, with lower doses blocking gap junction hemichannel opening and higher doses uncoupling gap junctions between cells. See, e.g., O'Carroll et al., 2008. With sustained low dose application there is also gradual loss of gap junction coupling, considered to be peptide interference with hemichannel docking (in parallel with gradual removal of existing gap junctions during normal turnover). Peptagon has proven to be effective in a number of in vitro, ex vivo and in vivo (animal) studies (see for example Davidson et al, 2012; Danesh-Meyer et al, 2012; O'Carroll et al, 2013).

In some embodiments, the hemichannel blockers, e.g., Cx43 hemichannel blockers, can comprise peptides. A hemichannel blocker peptide sequence can comprise, consist essentially of, or consist of, for example, one or more of the following sequences: SRPTEKT “Mod3” (SEQ ID NO:2), “Peptide 1” ADCFLSRPTEKT (SEQ ID NO:3), “Peptide 2” VACFLSRPTEKT (SEQ ID NO:4), “Peptide 11” VDCFLSRPTAKT (SEQ ID NO:5), “Peptide 12” VDCFLSRPTEAT (SEQ ID NO:6), “Peptide 5” VDCFLSRPTEKT (SEQ ID NO:1), “Mod1” CFLSRPTEKT (SEQ ID NO:7), “Mod2” LSRPTEKT (SEQ ID NO:8). In some embodiments, the carboxy-terminus can be modified. In some aspects, the carboxy-terminus modification can comprise n-alkyl chains which can optionally be further linked to hydrogen or other moieties. In some embodiments, the hemichannel blocker peptides can include or exclude any of the peptides listed above or disclosed herein.

In one aspect, the invention relates to the use of pharmaceutical compositions, alone or within kits, packages or other articles of manufacture, in methods for treating diseases, disorders, or conditions noted herein, as well as those characterized, for example, by decreased or disordered RPE integrity, BRB integrity, tight junction integrity and/or increased or disordered production of of type IV collagen. The methods herein provide for treatment of a subject with a hemichannel blocker in an amount sufficient for the modulation of RPE integrity, BRB integrity, tight junction integrity and/or modulation of type IV collagen production, amongst other things, as noted herein. In some preferred aspects, the hemichannel blocker is a connexin 43 hemichannel blocker. In other aspects, the hemichannel blocker is a connexin 36 hemichannel blocker. In still other aspects, the hemichannel blocker is a connexin 37 hemichannel blocker. In other aspects, the hemichannel blocker is a connexin 45 hemichannel blocker. Blockers of other connexin hemichannels are within the invention, as noted.

In some embodiments “promoiety” refers to a species acting as a protecting group which masks a functional group within an active agent, thereby converting the active agent into a pro-drug. Typically, the promoiety will be attached to the drug via bond(s) that are cleaved by enzymatic or non-enzymatic means in vivo, thereby converting the pro-drug into its active form. In some embodiments the promoiety may also be an active agent. In some embodiments the promoiety may be bound to a hemichannel blocker. In some embodiments the promoiety may be bound to any of a peptide or peptidomimetic or small molecule hemichannel blocker, for example. In some embodiments the promoeity may be bound to a compound of Formula I. In some embodiments the pro-drug may be another hemichannel compound, e.g., a compound described in Green et al., US Pat. App. Publication No. 20160177298; Savory, et al., US Pat. App. Publication No. 20160318891; or Savory, et al., US Pat. App. Publication No. 20160318892.

In some aspects, hemichannel blockers include, for example, antibodies or antibody fragments, nanobodies, peptide or peptidomimetics, recombinant fusion proteins, aptamers, small molecules, or single chain variable fragments (scFv) that bind to a connexin hemichannel, and others noted herein. In one presently preferred embodiment, the connexin hemichannel is a Cx43 hemichannel.

In other embodiments, the hemichannel blockers are connexin 43 peptides or peptidomimetics, sometimes referred to as hemichannel blocking peptides or peptidomimetics, and include modified or unmodified Cx peptides or peptidomimentics comprising, consisting essentially of, or consisting of connexin extracellular domains, transmembrane regions, and connexin carboxy-terminal peptides. In some aspects, the therapeutically effective modified or unmodified peptide or peptidomimetic comprises a portion of an extracellular or transmembrane domain of a connexin 43 or connexin 45. The protein sequence of connexin 43 is shown below.

```
Connexin 43
(SEQ ID NO: 9)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
              20 25 30
Arg Ile Leu Leu Leu Gly Thr Ala Val Glu Ser Ala Trp Gly Asp Glu
        35 40 45
Gln Ser Ala Phe Arg Cys Asn Thr Gln Gln Pro Gly Cys Glu Asn Val
  50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                      85 90 95
Phe Tyr Val Met Arg Lys Glu Glu Lys Leu Asn Lys Lys Glu Glu Glu
            100 105 110
Leu Lys Val Ala Gln Thr Asp Gly Val Asn Val Asp Met His Leu Lys
        115 120 125
Gln Ile Glu Ile Lys Lys Phe Lys Tyr Gly Ile Glu Glu His Gly Lys
    130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                  165 170 175
Tyr Gly Phe Ser Leu Ser Ala Val Tyr Thr Cys Lys Arg Asp Pro Cys
              180 185 190
Pro His Gln Val Asp Cys Phe Leu Ser Arg Pro Thr Glu Lys Thr Ile
          195 200 205
Phe Ile Ile Phe Met Leu Val Val Ser Leu Val Ser Leu Ala Leu Asn
    210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                  245 250 255
Ala Lys Asp Cys Gly Ser Gln Lys Tyr Ala Tyr Phe Asn Gly Cys Ser
            260 265 270
Ser Pro Thr Ala Pro Leu Ser Pro Met Ser Pro Pro Gly Tyr Lys Leu
          275 280 285
Val Thr Gly Asp Arg Asn Asn Ser Ser Cys Arg Asn Tyr Asn Lys Gln
  290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                  325 330 335
Phe Pro Asp Asp Asn Gln Asn Ser Lys Lys Leu Ala Ala Gly His Glu
            340 345 350
Leu Gln Pro Leu Ala Ile Val Asp Gln Arg Pro Ser Ser Arg Ala Ser
        355 360 365
Ser Arg Ala Ser Ser Arg Pro Arg Pro Asp Asp Leu Glu Ile
    370 375 380
```

Table 1 shows extracellular loops for connexin 43 and connexin 45. In some embodiments, the therapeutically effective modified or unmodified peptide or peptidomimetic comprises a portion of the E2 extracellular domain of a connexin (extracellular loop 2), such as connexin 43 or connexin 45, preferably connexin 43. In some embodiments, the therapeutically effective modified or unmodified peptide or peptidomimetic comprises a portion of the C-terminal domain of a connexin, such as connexin 43 or connexin 45, preferably connexin 43. If a peptide or peptidomimetic blocker comprises a portion of an intracellular domain of a connexin, the peptide may, in some embodiments, be conjugated to a cell internalization transporter and may, in some instances, block zona occludens (ZO-1) binding to connexin 43.

TABLE 1
Extracellular loops for connexin 43
and connexin 45

| | | |
|---|---|---|
| E1 | | |
| huCxn43 | ESAWGDEQSAFRCNTQQPGCENVCYD | (SEQ ID NO: 10) |
| | KSFPISHVR | |
| huCx45 | GESIYYDEQSKFVCNTEQPGCENVCY | (SEQ ID NO: 11) |
| | DAFAPLSHVR | |
| E2 | | |
| huCxn43 | LLIQWYIYGFSLSAVYTCKRDPCPHQ | (SEQ ID NO: 12) |
| | VDCFLSRPTEKT | |
| huCx45 | LIGQYFLYGFQVHPFYVCSRLPCHPK | (SEQ ID NO: 13) |
| | IDCFISRPTEKT | |

Sequences of the E2 domain of different connexin isotypes are shown with amino acids homologous to peptide SEQ ID NO:14 and peptide SEQ ID NO:15 shown in bold in Table 2. Note that last 4 amino acids of peptide SEQ ID NO:15 are part of the fourth membrane domain.

Table 2 provides the extracellular domain for connexin family members which can be used to prepare peptide hemichannel blockers described herein. The peptides and provided in Table 2, and fragments thereof, are used as peptide hemichannel blockers in certain non-limiting embodiments. In other non-limiting embodiments, hemichannel blocker peptides comprising, consisting essentially of, or consisting from about 8 to about 15, or from about 11 to about 13 amino contiguous amino acids of the peptides in this Table are peptide hemichannel blockers of the invention. In other embodiments, conservative amino acid changes are made to the peptides or fragments thereof.

TABLE 2
Extracellular domains

| | | |
|---|---|---|
| peptide | VDCFLSRPTEKT | (SEQ ID NO: 1) |
| peptide | SRPTEKTIFII | (SEQ ID NO: 16) |
| huCxn43 | LLIQWYIYGFSLSAVYTCKRDPCPHQ | (SEQ ID NO: 17) |
| | VDCFLSRPTEKTIFII | |
| huCx45 | LIGQYFLYGFQVHPFYVCSRLPCHPK | (SEQ ID NO: 18) |
| | IDCFISRPTEKTIFLL | |

Other peptide hemichannel blockers are from the cytoplasmic loop of connexin 43 (amino acids 119-144) L2 peptide and subparts of the L2 peptide of connexin 43. In some embodiments, these peptides may include or exclude, for example, the nine amino acid sequence of Gap 19, KQIEIKKFK (SEQ ID NO:19); the native Gap19 sequence, DGVNVEMHLKQIEIKKFKYGIEEHGK (SEQ ID NO:20); the His144→Glu L2 derivative of Gap19, as reported by Shibayama (Shibayama, J. et al., Biophys. J. 91, 405404063, 2006), DGVNVEMHLKQIEIKKFKYGIEEQGK (SEQ ID NO:21); the TAT-Gap19 sequence, YGRKKRRQRRRKQIEIKKFK (SEQ ID NO:22); the SH3 binding domain, CSSPTAPLSPMSPPGYK (SEQ ID NO:23), or subpart thereof PTAPLSPMSPP (SEQ ID NO:24); the C-terminal sequence of the CT9 or CT10 peptide, with or without a TAT leader sequence to increase cell penetration, RPRDDEI (SEQ ID NO:25), SRPRDDLEI (SEQ ID NO:26), YGRKKRRQRRRSRPRDDEI (SEQ ID NO:27), or YGRKKRRQRRRRPRDDEI (SEQ ID NO:28). Other peptidomimetic sequences that can be included or excluded in the compositions for use in the methods, kits or articles of manufacture disclosed herein are those reported by Dhein (Dhein, S., Naunyn-Schmiedeberg's Arch. Pharm., 350: 174-184, 1994); the AAP10 peptide, H₂N-Gly-Ala-Gly-4Hyp-Pro Tyr-CONH₂ (SEQ ID NO:29), and the ZP123 peptide (rotigapeptide), Ac-D-Tyr-Pro-D-4Hyp-Gly-D-Ala-Gly-NH₂ (SEQ ID NO: 91), (Dhein, S., et al. Cell Commun. Adhes. 10, 371-378, 2013). Rotigapeptide is comprised of the D-form of the peptides for enhanced efficacy over the native L-form of the peptide.

Exemplary connexin 43 (Cx43) or Cx26, Cx30, Cx30.3, Cx31, Cx31.1, Cx32, Cx36, Cx37, Cx40.1, Cx43, Cx46, Cx46.6, or Cx40 peptide blockers that may be included or excluded in certain embodiments of this disclosure are provided in Table 3 below (E2 and T2 refer to the location of a peptide in, for example, the second extracellular domain or the second transmembrane domain).

TABLE 3

| SEQ ID NO: | Identifier | Sequence |
|---|---|---|
| SEQ ID NO: 30 | CXT 2 | PSSRASSRASSRPRPDDLEI |
| SEQ ID NO: 31 | CXT 1 | RPRPDDLEI |
| SEQ ID NO: 32 | CXT 3 | RPRPDDLEV |
| SEQ ID NO: 33 | CXT 4 | RPRPDDVPV |
| SEQ ID NO: 34 | CXT 5 | KARSDDLSV |
| SEQ ID NO: 35 | hCx40 | QKPEVPNGVSPGHRLPHGYHSDKRRLSKASSKARS |
| | | DDLSV |
| SEQ ID NO: 36 | Antp/CXT 2 | RQPKIWFPNRRKPWKKPSSRASSRASSRPRPDDLEI |
| SEQ ID NO: 37 | Antp/CXT 2 | RQPKIWFPNRRKPWKKPSSRASSRASSRPRPDDLEI |
| SEQ ID NO: 38 | Antp/CXT 1 | RQPKIWFPNRRKPWKKRPRPDDLEI |
| SEQ ID NO: 39 | Antp/CXT 3 | RQPKIWFPNRRKPWKKRPRPDDLEV |
| SEQ ID NO: 40 | Antp/CXT 4 | RQPKIWFPNRRKPWKKRPRPDDVPV |
| SEQ ID NO: 41 | Antp/CXT 5 | RQPKIWFPNRRKPWKKKARSDDLSV |
| SEQ ID NO: 42 | conservative Cxn43 | RPKPDDLDI |
| | variant | |
| SEQ ID NO: 43 | HIV-Tat/CXT 1 | GRKKRRQRPPQRPRPDDLEI |
| SEQ ID NO: 44 | Penetratin/CXT 1 | RQIKIWFQNRRMKWKKRPRPDDLEI |
| SEQ ID NO: 45 | Antp-3A/CXT 1 | RQIAIWFQNRRMKWAARPRPDDLEI |
| SEQ ID NO: 46 | Tat/CXT 1 | RKKRRQRRRRPRPDDLEI |
| SEQ ID NO: 47 | Buforin II/Vnrs 1 | TRSSRAGLQFPVGRVHRLLRKRPRPDDLEI |
| SEQ ID NO: 48 | Transportan/CXT 1 | GWTLNSAGYLLGKINKALAALAKKILRPRPDDLEI |
| SEQ ID NO: 49 | MAP/CXT 1 | KLALKLALKALKAALKLARPRPDDLEI |
| SEQ ID NO: 50 | K-FGF/CXT 1 | AAVALLPAVLLALLAPRPRPDDLEI |
| SEQ ID NO: 51 | Ku70/CXT 1 | VPMLKPMLKERPRPDDLEI |
| SEQ ID NO: 52 | Prion/CXT 1 | MANLGYWLLALFVTMWTDVGLCKKRPKPRPRPD |
| | | DLEI |
| SEQ ID NO: 53 | pVEC/CXT 1 | LLIILRRRIRKQAHAHSKRPRPDDLEI |
| SEQ ID NO: 54 | Pep-1/CXT 1 | KETWWETWWTEWSQPKKKRKVRPRPDDLEI |
| SEQ ID NO: 55 | SynB1/CXT 1 | RGGRLSYSRRRFSTSTGRRPRPDDLEI |
| SEQ ID NO: 56 | Pep-7/CXT 1 | SDLWEMMMVSLACQYRPRPDDLEI |
| SEQ ID NO: 57 | HN-1/CXT 1 | TSPLNIHNGQKLRPRPDDLEI |
| SEQ ID NO: 1 | SEQ-pept5, or | VDCFLSRPTEKT |
| | Peptide 5 | |
| SEQ ID NO: 59 | SEQ-Gap27 | SRPTEKTIFII |
| SEQ ID NO: 60 | SEQ-Gap26 | VCYDKSFPISHVR |
| SEQ ID NO: 61 | SEQ-Mod1 | CFLSRPTEKT |
| SEQ ID NO: 62 | SEQ-Mod2 | LSRPTEKT |
| SEQ ID NO: 63 | SEQ-Mod3 | SRPTEKT |
| SEQ ID NO: 64 | SEQ-Mod4 | VDCFLSRPTE |
| SEQ ID NO: 65 | SEQ-Mod5 | VDCFLSRP |
| SEQ ID NO: 66 | SEQ-Mod6 | VDCFLS |
| SEQ ID NO: 67 | HIV-Tat/SEQ- | GRKKRRQRPPQVDCFLSRPTEKT |
| | pept5 | |
| SEQ ID NO: 68 | Penetratin/SEQ- | RQIKIWFQNRRMKWKKVDCFLSRPTEKT |
| | pept5 | |
| SEQ ID NO: 69 | Antp-3A/SEQ- | RQIAIWFQNRRMKWAAVDCFLSRPTEKT |
| | pept5 | |
| SEQ ID NO: 70 | Tat/SEQ-pept5 | RKKRRQRRRVDCFLSRPTEKT |
| SEQ ID NO: 71 | Buforin II/SEQ- | TRSSRAGLQFPVGRVHRLLRKVDCFLSRPTEKT |
| | pept5 | |
| SEQ ID NO: 72 | Transportan/SEQ- | GWTLNSAGYLLGKINKALAALAKKILVDCFLSRPT |
| | pept5 | EKT |
| SEQ ID NO: 73 | MAP/SEQ-pept5 | KLALKLALKALKAALKLAVDCFLSRPTEKT |
| SEQ ID NO: 74 | K-FGF/SEQ-pept5 | AAVALLPAVLLALLAPVDCFLSRPTEKT |
| SEQ ID NO:75 | Ku70/SEQ-pept5 | VPMLKPMLKEVDCFLSRPTEKT |
| SEQ ID NO: 76 | Prion/SEQ-pept5 | MANLGYWLLALFVTMWTDVGLCKKRPKPVDCFLS |
| | | RPTEKT |
| SEQ ID NO: 77 | pVEC/SEQ-pept5 | LLIILRRRIRKQAHAHSKVDCFLSRPTEKT |
| SEQ ID NO: 78 | Pep-1/SEQ-pept5 | KETWWETWWTEWSQPKKKRKVVDCFLSRPTEKT |
| SEQ ID NO: 79 | SynB1/SEQ-pept5 | RGGRLSYSRRRFSTSTGRVDCFLSRPTEKT |
| SEQ ID NO: 80 | Pep-7/SEQ-pept5 | SDLWEMMMVSLACQYVDCFLSRPTEKT |
| SEQ ID NO: 81 | HN-1/SEQ-pept5 | TSPLNIHNGQKLVDCFLSRPTEKT |
| SEQ ID NO: 82 | SEQ M3E2 | FEVAFLLIQWI |
| SEQ ID NO: 83 | SEQ E2a | LLIQWYIGFSL |
| SEQ ID NO: 84 | SEQ E2b | SLSAVYTCKRDPCPHQ |
| SEQ ID NO: 85 | SEQ E2c | SRPTEKTIFII |
| SEQ ID NO: 86 | SEQ M1E1 | LGTAVESAWGDEQ |
| SEQ ID NO: 87 | SEQ Ela | QSAFRCNTQQPG |
| SEQ ID NO: 88 | SEQ Elb | QQPGCENVCYDK |
| SEQ ID NO: 89 | SEQ Elc | VCYDKSFPISHVR |
| SEQ ID NO: 90 | SEQ E2d | KRDPCHQVDCFLSRPTEK |
| SEQ ID NO: 3 | Peptide 1 | ADCFLSRPTEKT |
| SEQ ID NO: 4 | Peptide 2 | VACFLSRPTEKT |
| SEQ ID NO: 5 | Peptide 11 | VDCFLSRPTAKT |
| SEQ ID NO: 6 | Peptide 12 | VDCFLSRPTEAT |
| SEQ ID NO: 19 | Gap 19-subpart | KQIEIKKFK |
| SEQ ID NO: 20 | Gap 19-full | DGVNVEMHLKQIEIKKFKYGIEEHGK |
| SEQ ID NO: 21 | Gap 19-deny | DGVNVEMHLKQIEIKKFKYGIEEQGK |
| SEQ ID NO: 22 | TAT-Gap19 | YGRKKRRQRRRKQIEIKKFK |
| SEQ ID NO: 23 | SH3-full | CSSPTAPLSPMSPPGYK |
| SEQ ID NO: 24 | SH3-subpart | PTAPLSPMSPP |
| SEQ ID NO: 25 | C-terminus CT9 | RPRDDEI |
| SEQ ID NO: 27 | C-terminus CT9- | YGRKKRRQRRRSRPRDDEI |
| | TAT | |
| SEQ ID NO: 26 | C-terminus CT10 | SRPRDDLEI |
| SEQ ID NO: 28 | C-terminus CT10- | YGRKKRRQRRRRPRDDEI |
| | TAT | |
| SEQ ID NO: 29 | AAP10 | H₂N-Gly-Ala-Gly-4Hyp-Pro Tyr-CONH₂ |
| SEQ ID NO: 91 | ZP123 | Ac-D-Tyr-Pro-D-4Hyp-Gly-D-Ala-Gly-NH₂ |
| SEQ ID NO: 92 | p1s1/SEQ-pept5 | RVIRVWFQNKRCKDKKVDCFLSRPTEKT |
| SEQ ID NO: 93 | MGB Peptide P- | GALFLGFLGAAGSTMGAWSQPKKKRKVVDCFLSR |
| | beta/SEQ-pept5 | PTEKT |
| SEQ ID NO: 94 | MGB Peptide P- | GALFLAFLAAALSLMGLWSQPKKKRRVVDCFLSRP |
| | alpha/SEQ-pept5 | TEKT |
| SEQ ID NO: 95 | huCx26 | MYVFYVMYDGFSMQRLVKCNAWPCPNTVDCFVS |
| | | RPTEKT |
| SEQ ID NO: 96 | huCx30 | MYVFYFLYNGYHLPWVLKCGIDPCPNLVDCFISRP |
| | | TEKT |
| SEQ ID NO: 97 | huCx30.3 | LYIFHRLYKDYDMPRVVACSVEPCPHTVDCYISRPT |
| | | EKK |
| SEQ ID NO: 98 | huCx31 | LYLLHTLWHGFNMPRLVQCANVAPCPNIVDCYIAR |
| | | PTEKK |
| SEQ ID NO: 99 | huCx31.1 | LYVFHSFYPKYILPPVVKCHADPCPNIVDCFISKPSE |
| | | KN |
| SEQ ID | huCx32 | MYVFYLLYPGYAMVRLVKCDVYPCPNTVDCFVSR |
| NO: 100 | | PTEKT |
| SEQ ID | huCx36 | LYGWTMEPVFVCQRAPCPYLVDCFVSRPTEKT |
| NO: 101 | | |
| SEQ ID | huCx37 | LYGWTMEPVFVCQRAPCPYLVDCFVSRPTEKT |
| NO: 102 | | |
| SEQ ID | huCx40.1 | GALHYFLFGFLAPKKFPCTRPPCTGVVDCYVSRPTE |
| NO: 103 | | KS |
| SEQ ID | huCx43 | LLIQWYIYGFSLSAVYTCKRDPCPHQVDCFLSRPTE |
| NO: 104 | | KT |
| SEQ ID | huCx46 | IAGQYFLYGFELKPLYRCDRWPCPNTVDCFISRPTE |
| NO: 105 | | KT |
| SEQ ID | huCx46.6 | LVGQYLLYGFEVRPFFPCSRQPCPHVVDCFVSRPTE |
| NO: 106 | | KT |
| SEQ ID | huCx40 | IVGQYFIYGIFLTTLHVCRRSPCPHPVNCYVSRPTEK |
| NO: 107 | | N |

In some embodiments the connexin 43 blocker may comprise, for example, a peptide or peptidomimetic comprising, consisting essentially of, or consisting of, for example SEQ ID NO:2 (SRPTEKT). The peptide or peptidomimetic may also comprise, for example SEQ ID NO:1 (VDCFLSRPTEKT). The peptide may contain one or more modified amino acids, amino acid analogs, or may be otherwise modified to improve bioavailability or to increase penetration across the cell membrane. For example, SEQ ID NO:1 may be modified to obtain SEQ ID NOS:20-25 and 27. In some aspects the peptide or peptidomimetic comprising, consisting essentially of, or consisting of for example SEQ ID NO:2(SRPTEKT) or SEQ ID NO:1(VDCFLSRPTEKT) comprises from 7 to 40 amino acids or amino acid analogues and does not comprise a C-terminal peptide. In some embodiments, the peptides may also be used as promoieties.

In some aspects, the connexin 45 blockers can be peptide or peptidomimetics comprising, consisting essentially of, or consisting of portions of the connexin 45 protein that antagonize or inhibit or block connexin-connexin interactions. Exemplary peptide sequences for connexin 45 peptides and peptidomimetic blockers are provided in Table 4.

TABLE 4
Sequences of Sample Connexin 45 Blocker
Peptides or Peptidomimetics

| SEQ ID NO. | Sequence |
|---|---|
| SEQ ID | LTAVGGESIYYDEQSKFVCNTEQPGCENVCYDAFAPLSH |
| NO: 108 | VRFWVFQ |
| SEQ ID | LTAVGGESIYYDEQS |
| NO: 109 | |
| SEQ ID | DEQSKFVCNTEQP |
| NO: 110 | |
| SEQ ID | TEQPGCENVCYDA |
| NO: 111 | |
| SEQ ID | VCYDAFAPLSHVR |
| NO: 112 | |
| SEQ ID | APLSHVRFWVFQ |
| NO: 113 | |
| SEQ ID | FEVGFLIGQYFLYGFQVHPFYVCSRLPCHPKIDCFISRPT |
| NO: 114 | EKTIFLL |
| SEQ ID | FEVGFLIGQYF |
| NO: 115 | |
| SEQ ID | LIGQYFLYGFQV |
| NO: 116 | |
| SEQ ID | GFQVHPFYVCSRLP |
| NO: 117 | |
| SEQ ID | SRLPCHPKIDCF |
| NO: 118 | |
| SEQ ID | IDCFISRPTEKT |
| NO: 119 | |
| SEQ ID | SRPTEKTIFLL |
| NO: 120 | |
| SEQ ID | SRPTEKTIFII |
| NO: 121 | |
| SEQ ID | YVCSRLPCHP |
| NO: 122 | |
| SEQ ID | QVHPFYVCSRL |
| NO: 123 | |
| SEQ ID | FEVGFLIGQYFLY |
| NO: 124 | |
| SEQ ID | GQYFLYGFQVHP |
| NO: 125 | |
| SEQ ID | GFQVHPFYVCSR |
| NO: 126 | |
| SEQ ID | AVGGESIYYDEQ |
| NO: 127 | |
| SEQ ID | YDEQSKFVCNTE |
| NO: 128 | |
| SEQ ID | NTEQPGCENVCY |
| NO: 129 | |
| SEQ ID | CYDAFAPLSHVR |
| NO: 130 | |
| SEQ ID | FAPLSHVRFWVF |
| NO: 131 | |
| SEQ ID | LIGQY |
| NO: 132 | |
| SEQ ID | QVHPF |
| NO: 133 | |
| SEQ ID | YVCSR |
| NO: 134 | |
| SEQ ID | SRLPC |
| NO: 135 | |
| SEQ ID | LPCHP |
| NO: 136 | |
| SEQ ID | GESIY |
| NO: 137 | |
| SEQ ID | YDEQSK |
| NO: 138 | |
| SEQ ID | SKFVCN |
| NO: 139 | |
| SEQ ID | TEQPGCEN |
| NO: 140 | |
| SEQ ID | VCYDAFAP |
| NO: 141 | |
| SEQ ID | LSHVRFWVFQ |
| NO: 142 | |
| SEQ ID | LIQYFLYGFQVHPF |
| NO: 143 | |
| SEQ ID | VHPFYCSRLPCHP |
| NO: 144 | |
| SEQ ID | VGGESIYYDEQSKFVCNTEQPG |
| NO: 145 | |
| SEQ ID | TEQPGCENVCYDAFAPLSHVRF |
| NO: 146 | |
| SEQ ID | AFAPLSHVRFWVFQ |
| NO: 147 | |
| SEQ ID | IDCFISRPTEKTIFLL |
| NO: 148 | |
| SEQ ID | DCFISRPTEKT |
| NO: 149 | |
| SEQ ID | SRPTEKT |
| NO: 150 | |
| SEQ ID | LIGQYFLYGFQVHPFYVCSRLPCHPKIDCFISRPTEKT |
| NO: 151 | |

In some embodiments the connexin 45 blocker may comprise, for example, a peptide or peptidomimetic comprising, consisting essentially of, or consisting of a portion of the E2 or C terminal domain of connexin 45, for example, comprising, consisting essentially of, or consisting of SEQ ID NO:150 (SRPTEKT). The peptide or peptidomimetic may also comprise, for example SEQ ID NO:149 (DCFISRPTEKT). In some embodiments the peptides may only be 3 amino acids in length, including SRL, PCH, LCP, CHP, WY, SKF, QPC, VCY, APL, HVR, or longer.

In some aspects, the connexin 40 hemichannel blockers can be peptide or peptidomimetics comprising, consisting essentially of, or consisting of portions of the connexin 40 protein. In some embodiments, the connexin 43 blocker may comprise, consist essentially of, or consist of, for example, SEQ ID NO:2 (SRPTEKT), SEQ ID NO:1 (VDCFLSRPTEKT), or SEQ ID NO:1 conjugated to two dodecyl groups at the N-terminus, through a linker. The peptide may contain one or more modified amino acids, amino acid analogs, or may be otherwise modified, for example, conjugated or bound to cell internalization transporter.

In another non-limiting but preferred embodiment, hemichannel blocker comprises a peptide comprising, consisting essentially of, or consisting of an amino acid sequence corresponding to a portion of a transmembrane region of a connexin, such as Cx43 or Cx45, or Cx26, Cx37, or Cx40. In particular non-limiting embodiments, the anti-connexin compound is a peptide having an amino acid sequence that comprises a peptide having an amino acid sequence that comprises about 3 to about 30 contiguous amino acids of the connexin, e.g., connexin 43 or 45 protein sequence, about 5 to about 20 contiguous amino acids of the connexin protein sequence, a peptide having an amino acid sequence that comprises about 8 to about 15 contiguous amino acids of the connexin protein sequence, or a peptide having an amino acid sequence that comprises about 11, 12, or 13 contiguous amino acids of the connexin protein sequence. Other non-limiting embodiments include an anti-connexin compound that is a peptide having an amino acid sequence that comprises at least about 5, 6, 7, 8, 9, 10, 11, 12, 13, 14, 15, 20, 25, or 30 contiguous amino acids of the connexin protein sequence. In some aspects, the hemichannel blocker can include or exclude any of the foregoing.

In other anti-connexin compounds, mimetic peptides are based on the extracellular domains of connexin 43 corresponding to the amino acids at positions 37-76 and 178-208 of connexin 43 protein sequence. Thus, certain peptides described herein have an amino acid sequence corresponding to the regions at positions 37-76 and 178-208 of the connexin 43 protein sequence. The peptides need not have an amino acid sequence identical to those portions of the connexin 43 protein sequence, and conservative amino acid changes may be made such that the peptides retain binding activity or functional activity in the assays described herein and otherwise known in the art. In other embodiments, mimetic peptides are based on peptide target regions within the connexin protein other than the extracellular domains (e.g., the portions of the connexin 43 protein sequence not corresponding to positions 37-76 and 178-208).

In a non-limiting but preferred embodiment, a hemichannel blocker comprises, consists essentially of, or consists of a peptide comprising, consisting essentially of, or consisting of an amino acid sequence corresponding to a portion of a transmembrane region of connexin 45 or a C-terminal region of connexin 45. In particular non-limiting embodiments, for example, the anti-connexin compound is a peptide having an amino acid sequence that comprises about 3 to about 30 contiguous amino acids of the known connexin 45 sequence, a peptide having an amino acid sequence that comprises about 5 to about 20 contiguous amino acids of the known connexin 45 sequence, a peptide having an amino acid sequence that comprises about 8 to about 15 contiguous amino acids of the known connexin 45 sequence, or a peptide having an amino acid sequence that comprises about 11, 12, or 13 contiguous amino acids of the known connexin 45 sequence. Other non-limiting embodiments include an anti-connexin compound that is a peptide having an amino acid sequence that comprises at least about 3, 4, 5, 6, 7, 8, 9, 10, 11, 12, 13, 14, 15, 20, 25, or 30 contiguous amino acids of the known connexin 45 sequence. In certain anti-connexin compounds provided herein, mimetic peptides are based on the extracellular domains of connexin 45 corresponding to the amino acids at positions 46-75 and 199-228 of the known connexin 45 sequence. Thus, certain peptide described herein have an amino acid sequence corresponding to the regions at positions 46-75 and 199-228 of the known connexin 45 sequence. The peptides need not have an amino acid sequence identical to those portions of the known connexin 45 sequence. Conservative amino acid changes may be made such that the peptides retain binding activity or functional activity in the assays described herein and otherwise known in the art. In other embodiments, mimetic peptides are based on peptide target regions within the connexin protein other than the extracellular domains (e.g., portions of the known connexin 45 sequence not corresponding to positions 46-75 and 199-228). WO2006/134494, disclosing various connexin sequences is incorporated in its entirety by reference. In some aspects, the hemichannel blocker can include or exclude any of the foregoing.

### Other Connexin Hemichannel Blockers

Hemichannel blockers, for example, connexin 36, 37, 43 or 45 blockers, including peptides, peptidomimetics, antibodies, antibody fragments, and the like, are also suitable hemichannel blockers. Exemplary hemichannel blockers may include, without limitation, polypeptides (e.g. antibodies, binding fragments thereof, and synthetic constructs), and other gap junction blocking agents, and gap junction protein phosphorylating agents. In some aspects the hemichannel blocker is a blocker of Cx26, Cx30, Cx31.1, Cx36, Cx37, Cx40, Cx43, Cx50, Cx57. Hemichannel blockers, for example, connexin 36, 37, 43 or 45 blockers include, for example, monoclonal antibodies, polyclonal antibodies, antibody fragments (including, for example, Fab, F(ab′)2 and Fv fragments; single chain antibodies; single chain Fvs; and single chain binding molecules such as those comprising, consisting essentially of, or consisting of, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, hemichannel, or associated molecules).

Binding molecules will generally have a desired specificity, including but not limited to binding specificity, and desired affinity. Affinity, for example, may be a Ka of greater than or equal to about 10⁴ M-1, greater than or equal to about 10⁶ M-1, greater than or equal to about 10⁷ M-1, greater than or equal to about 10⁸ M-1. Affinities of even greater than about 10⁸ M-1 are suitable, such as affinities equal to or greater than about 10⁹ M-1, about 101⁰ M-1, about 10¹¹ M-1, and about 10¹² M-1. Affinities of binding proteins according to the present invention can be readily determined using conventional techniques, for example those described by Scatchard et al., (1949) Ann. N.Y. Acad. Sci. 51: 660.

Exemplary compounds used for closing gap junctions (e.g. phosphorylating connexin 43 tyrosine and/or serine residue) have been reported in U.S. Pat. Nos. 7,153,822 and 7,250,397. Exemplary peptides and peptidomimetics are reported in Green et al., WO2006134494. See also WO2006069181 and WO2003032964. Examples of other agents used for closing gap junctions include anti-connexin agents, for example anti-connexin polynucleotides (for example, connexin inhibitors such as alpha-1 connexin oligodeoxynucleotides), anti-connexin peptides (for example, antibodies and antibody binding fragments) and peptidomimetics (for example, alpha-1 anti-connexin peptides or peptidomimetics), gap junction closing or blocking compounds, hemichannel closing or blocking compounds, and connexin carboxy-terminal polypeptides, e.g., polypeptides that are reported to bind to ZO-1 or a ZO-1 binding site.

Other hemichannel blockers useful in the invention also include, or may be combined with, compounds that block connexin hemichannels but maintain connexin gap junction function. For example, the linear peptide RRNYRRNY (SEQ ID NO: 174), the cyclic peptide CyRP-71 and the peptidomimetic molecule ZP2519 were demonstrated to target the Cx43 carboxy-terminal domain and to prevent Cx43-based gap junction closure under low pH conditions (Verma V, et al. Design and characterization of the first peptidomimetic molecule that prevents acidification-induced closure of cardiac gap junctions. Heart Rhythm 7:1491-1498 (2010); Verma V, et al. Novel pharmacophores of connexin43 based on the “RXP” series of Cx43-binding peptides. Circ. Res. 105:176-184 (2009)). These substances are of potential translational value for preventing gap junction closure. Moreover, these molecules are potential hemichannel blockers and may thus have two-sided actions directed at preventing gap junction closure as well as inhibiting hemichannel opening.

Anti-connexin agents include peptides having an amino acid sequence that comprises about 5 to 20 contiguous amino acids of a connexin protein such as connexin 43 (SEQ.ID.NO:19), peptides having an amino acid sequence that comprises about 8 to 15 contiguous amino acids of connexin 43, or peptides having an amino acid sequence that comprises about 11 to 13 contiguous amino acids of connexin 43. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of connexin 43. Other anti-connexin 43 blockers comprise the extracellular domains of connexin 43, for example, peptide or peptidomimetic comprising, consisting essentially of, or consisting of SRPTEKT (SEQ ID NO: 2) or VDCFLSRPTEKT (SEQ ID NO: 1). Other anti-connexin 43 blockers comprise the C-terminus region of connexin 43, see WO2006/069181, or modified versions thereof.

### Peptide Chemistry Modifications

In certain embodiments, the connexin 43 blocker peptides of the present invention can be linked at the amino or carboxy terminus to a cellular internalization transporter. The cellular internalization transporter linked to the connexin 43 blocker peptides of the present invention may be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (BisGuanidinium-Tren-Cholesterol).

Other sequences of exemplary cellular internalization peptides are provided in Table 5 below.

TABLE 5

| SEQ ID NO. | Identifier | Sequence |
|---|---|---|
| SEQ ID NO: 152 | ANTP | RQPKIWFPNRRKPWKK |
| SEQ ID NO: 153 | HIV-TAT | GRKKRRQRPPQ |
| SEQ ID NO: 154 | Transportan | GWTLNSAGYLLGKINKALAALAKKIL |
| SEQ ID NO: 155 | Buforin II | TRSSRAGLQFPVGRVHRLLRK |
| SEQ ID NO: 156 | Tat | RKKRRQRRR |
| SEQ ID NO: 157 | Penetratin | RQIKIWFQNRRMKWKK |
| SEQ ID NO: 158 | MAP | KLALKLALKALKAALKLA |
| SEQ ID NO: 159 | K-FGF | AAVALLPAVLLALLAP |
| SEQ ID NO: 160 | Ku70 | VPMLKPMLKE |
| SEQ ID NO: 161 | Prion | MANLGYWLLALFVTMWTDVGLCKKRPKP |
| SEQ ID NO: 162 | pVEC | LLIILRRRIRKQAHAHSK |
| SEQ ID NO: 163 | Pep-1 | KETWWETWWTEWSQPKKKRRV |
| SEQ ID NO: 164 | SynB1 | RGGRLSYSRRRFSTSTGR |
| SEQ ID NO: 165 | Pep-7 | SDLWEMMMVSLACQY |
| SEQ ID NO: 166 | HN-1 | TSPLNIHNGQKL |
| SEQ ID NO: 167 | plsl | RVIRVWFQNKRCKDKK |
| SEQ ID NO: 168 | MGB Peptide P- | GALFLGFLGAAGSTMGAWSQPKKKRKV |
| | beta | |
| SEQ ID NO: 169 | MGB Peptide P- | GALFLAFLAAALSLMGLWSQPKKKRRV |
| | alpha | |
| SEQ ID NO: 170 | From N-terminal | LCLRPVG |
| | region of the X- | |
| | protein of the | |
| | hepatitis B virus) | |

In one embodiment of the present invention, the amino acid sequence of the connexin 43 blocker peptides can be selected from the group consisting of any peptide SEQ ID listed herein, or a conservative variant thereof. In a further embodiment of the present invention, the connexin 43 blocker peptides can comprise, consist essentially of, or consist of, the amino acid sequence of SEQ ID NO:30-90. In another embodiment of the present invention, the connexin 43 blocker peptide further comprises a cellular internalization transporter. In a further embodiment, the connexin 43 hemichannel blocker peptide can be linked at the amino terminus to the cellular internalization transporter.

When specific proteins are referred to herein, derivatives, variants, and fragments are contemplated. Protein derivatives and variants are well understood to those of skill in the art and can involve amino acid sequence modifications. For example, amino acid sequence modifications can fall into one or more of three classes: insertional, substitutional or deletional variants. Insertions include amino and/or carboxyl terminal fusions as well as intrasequence insertions of single or multiple amino acid residues. Insertions can be smaller insertions than those of amino or carboxyl terminal fusions, for example, on the order of one to four residues. Deletions are characterized by the removal of one or more amino acid residues from the protein sequence(s). Substitutions, deletions, insertions or any combination thereof may be combined to arrive at a final construct. Substitutional variants are those in which at least one residue has been removed and a different residue inserted in its place. Such substitutions are referred to as conservative substitutions. The replacement of one amino acid residue with another that is biologically and/or chemically similar is known to those skilled in the art as a conservative substitution. A conservative substitution could replace one hydrophobic residue for another, or one polar residue for another. Conservatively substituted variations of each explicitly disclosed sequence are included within the peptides provided herein. Conservative substitutions typically have little to no impact on the biological activity of a resulting polypeptide. A conservative substitution can be an amino acid substitution in a peptide that does not substantially affect the biological function of the peptide. A peptide can include one or more amino acid substitutions, from 2-10 conservative substitutions, 2-5 conservative substitutions, or 4-9 conservative substitutions.

### Chemical Structure Modification

In certain embodiments, the chemical structure of the hemichannel blocker peptides or peptidomimetics can be synthetically modified to increase activity or half-life. For example, the peptide or peptidomimetic may be modified by conjugating the peptide to a hydrophobic compound, in some embodiments, through a linker moiety. The hydrophobic compound may be, for example, one or more n-alkyl groups, which may be, for example, C6-C14 alkyl groups. In some embodiments, the peptides may be conjugated at the N terminus to one or two dodecyl (C12) groups as described in Chen, Y S et al., J. Pharm. Sci., 102: 2322-2331 (2013), herein incorporated by reference. In one embodiment, the peptide sequence CFLSRPTEKT (SEQ ID NO: 7) or VDCFLSRPTEKT (SEQ ID NO: 1) can be conjugated to two dodecyl groups to create a modified peptide which can modulate connexin 43, “C12-C12-Cxn43 MP.” (SEQ ID NO:171). The resulting structure is shown below.

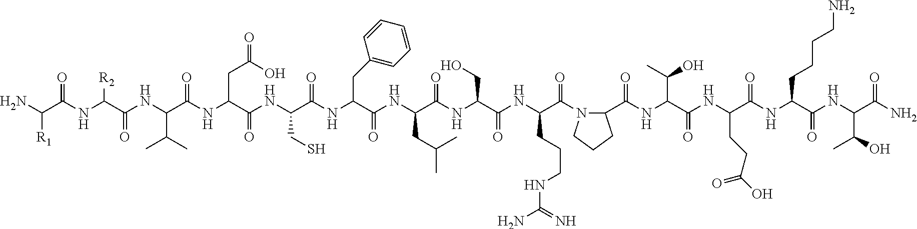

Structure: The structure of C12-C12-Cxn43 MP (SEQ ID NO:171). R₁ and R₂ can be hydrogen or alkyl groups (SEQ ID NO: 175). In some aspects, R₁=R₂=n-dodecyl chains.

### Chemical Delivery Modification

Hemichannel blockers useful in the present invention can also be formulated into microparticle (microspheres, Mps) or nanoparticle (nanospheres, Nps) formulations, or both. Particulate drug delivery systems include nanoparticles (1 to 1,000 nm) and microparticles (1 to 1,000 μm), which are further categorized as nanospheres and microspheres and nanocapsules and microcaps. In nanocapsules and microcapsules, the drug particles or droplets are entrapped in a polymeric membrane. Particulate systems have the advantage of delivery by injection, and their size and polymer composition influence markedly their biological behavior in vivo. Microspheres can remain in the vitreous for much longer periods of time than nanospheres, therefore, microparticles act like a reservoir after injection. Nanoparticles diffuse rapidly and are internalized in tissues and cells.

Assessing Hemichannel Blocker Activity Various methods may be used for assessing the activity or efficacy of hemichannel blockers. In one aspect of the invention, the effects of hemichannel blocker treatment in a subject is evaluated or monitored using assays for modulation of RPE integrity, BRB integrity, tight junction integrity and/or modulation of type IV collagen production, as described herein, by way of example.

The activity of hemichannel blockers may also be evaluated using certain biological assays. Effects of known or candidate hemichannel blockers on molecular motility can be identified, evaluated, or screened for using the methods described in the Examples below, or other art-known or equivalent methods for determining the passage of compounds through connexin hemichannels. Various methods are known in the art, including dye transfer experiments, for example, transfer of molecules labelled with a detectable marker, as well as the transmembrane passage of small fluorescent permeability tracers, which has been widely used to study the functional state of hemichannels. See, for example, Schlaper, K A, et al. Currently Used Methods for Identification and Characterization of Hemichannels. Cell Communication and Adhesion 15:207-218 (2008). In vivo methods may also be used. See, for example, the methods of Danesh-Meyer, H V, et al. Connexin43 mimetic peptide reduces vascular leak and retinal ganglion cell death following retinal ischaemia. Brain, 135:506-520 (2012); Davidson, J O, et al. (2012). Connexin hemichannel blockade improves outcomes in a model of fetal ischemia. Annals of Neurology 71:121-132 (2012).

One method for use in identifying or evaluating the ability of a compound to block hemichannels, comprises: (a) bringing together a test sample and a test system, said test sample comprising one or more test compounds, and said test system comprising a system for evaluating hemichannel block, said system being characterized in that it exhibits, for example, elevated transfer of a dye or labelled metabolite, for example, in response to the introduction of high glucose, hypoxia or ischemia to said system, a mediator of inflammation, or other compound or event that induces hemichannel opening, such as a drop in extracellular Ca²⁺; and, (b) determining the presence or amount of a rise in, for example, the dye or other labelled metabolite(s) in said system. Positive and/or negative controls may be used as well. Optionally, a predetermined amount of hemichannel blocker (e.g., Peptagon or Xiflam) may be added to the test system.

Preferably, hemichannel blockers, such as Peptagon and Xiflam, for example, exhibit activity in an in vitro assay on the order of less than about 1 to 5 nM, preferably less than about 10 nM and more preferably less than about 50 pM. In an in vivo assay these compounds preferably show hemichannel block at a concentration of less than about 10-100 micromolar (μM), and more preferably at a concentration of less than about 50 μM. Other hemichannel blockers may be within these ranges, and within a range of less than about 200 pM.

In one embodiment, a composition comprising, consisting essentially of, or consisting of one or more hemichannel blockers are administered. Hemichannel blocker(s) may be administered QD, BID, TID, QID, or in weekly doses, e.g., QIW, BIW QW. They may also be administered PRN (i.e., as needed), and HS (hora somni, i.e., at bedtime).

### Dosage Forms and Formulations and Administration

All descriptions with respect to dosing, unless otherwise expressly stated, apply to the hemichannel blockers of the invention.

The hemichannel blockers can be dosed, administered or formulated as described herein.

The hemichannel blockers can be administered to a subject in need of treatment. Thus, in accordance with the invention, there are provided formulations by which a connexin hemichannel, for example, a connexin 43 hemichannel or a connexin 45 hemichannel can be modulated to decrease its open probability in a transient and site-specific manner.

The hemichannel blockers may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a peptidomimetic or small molecule hemichannel blocker, for example, or dry mass of the preparation.

Administration of a hemichannel blocker to a subject may occur by any means capable of delivering the agents to a target site within the body of a subject. By way of example, a hemichannel blocker may be administered by one of the following routes: oral, topical, systemic (e.g., intravenous, intra-arterial, intra-peritoneal, transdermal, intranasal, or by suppository), parenteral (eg. intramuscular, subcutaneous, or intravenous or intra-arterial injection), by implantation, and by infusion through such devices as osmotic pumps, transdermal patches, and the like. Exemplary administration routes are also outlined in: Binghe, W. and B. Wang (2005). Drug delivery: principles and applications, Binghe Wang, Teruna Siahaan, Richard Soltero, Hoboken, N.J. Wiley-Interscience, c2005. In one embodiment, a hemichannel blocker is administered systemically. In another embodiment, a hemichannel blocker is administered orally. In another embodiment, a hemichannel blocker is administered topically or directly to an organ, cancer or tumor of interest, for example.

In some aspects, the hemichannel blocker may be provided as, or in conjunction with, an implant. In some aspects, may provide for sustained delivery. In some embodiments, a microneedle, needle, iontophoresis device or implant may be used for administration of the hemichannel blocker. The implant can be, for example, a dissolvable disk material such as that described in S. Pflugfelder et al., ACS Nano, 9 (2), pp 1749-1758 (2015). In some aspects, the hemichannel blockers, e.g. connexin 43 hemichannel blockers of this invention may be administered via intraventricular, and/or intrathecal, and/or extradural, and/or subdural, and/or epidural routes.

The hemichannel blocker may be administered once, or more than once, or periodically. It may also be administered PRN (as needed) or on a predetermined schedule or both. In some aspects, the hemichannel blocker is administered daily, weekly, monthly, bi-monthly or quarterly, or in any combination of these time periods. For example, treatment may be administered daily for a period, follow by weekly and/or monthly, and so on. Other methods of administering blockers are featured herein. In one aspect, a hemichannel blocker is administered to a patient at times on or between days 1 to 5, 10, 30, 45, 60, 75, 90 or day 100 to 180, in amounts sufficient to treat the patient.

A hemichannel blocker, such as Peptagon, for example, and/or an analogue or prodrug thereof, compounds of Formula I, for example Xiflam, and analogs or prodrugs of any of the foregoing compounds, or a compound of Formula II, may be administered alone or in combination with one or more additional ingredients and may be formulated into pharmaceutical compositions including one or more pharmaceutically acceptable excipients, diluents and/or carriers.

“Pharmaceutically acceptable diluents, carriers and/or excipients” is intended to include substances that are useful in preparing a pharmaceutical composition, may be co-administered with compounds of Formula I, for example Xiflam, and analogs of any of the foregoing compounds, or compounds of Formula II, while allowing it to perform its intended function, and are generally safe, non-toxic and neither biologically nor otherwise undesirable. Pharmaceutically acceptable diluents, carriers and/or excipients include those suitable for veterinary use as well as human pharmaceutical use. Suitable carriers and/or excipients will be readily appreciated by persons of ordinary skill in the art, having regard to the nature of compounds of Formula I, for example Xiflam, and analogs of any of the foregoing compounds. However, by way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also especially suitable for administration of agents.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as organ inserts, e.g., eye, or any other appropriate compositions. Persons of ordinary skill in the art to which the invention relates will readily appreciate the most appropriate dosage form having regard to the nature of the condition to be treated and the active agent to be used without any undue experimentation. It should be appreciated that one or more of hemichannel blocker, such as Peptagon, and/or an analogue thereof, compounds of Formula I, for example Xiflam, and analogs of any of the foregoing compounds, and/or a compound of Formula II, may be formulated into a single composition. In certain embodiments, preferred dosage forms include an injectable solution and an oral formulation.

Compositions useful in the invention may contain any appropriate level of hemichannel blocker, such as Peptagon, for example, and/or an analogue thereof, compounds of Formula I, for example Xiflam, and analogs of any of the foregoing compounds, and/or a compound of Formula II, having regard to the dosage form and mode of administration. However, by way of example, compositions of use in the invention may contain from approximately 0.1% to approximately 99% by weight, preferably from approximately 1% to approximately 60% of a hemichannel blocker, depending on the method of administration.

In addition to standard diluents, carriers and/or excipients, a composition in accordance with the invention may be formulated with one or more additional constituents, or in such a manner, so as to enhance the activity or bioavailability of hemichannel blocker, such as Peptagon, and/or an analogue thereof, compounds of Formula I, for example Xiflam, and analogs of any of the foregoing compounds, and/or a compound of Formula II, help protect the integrity or increase the half-life or shelf life thereof, enable slow release upon administration to a subject, or provide other desirable benefits, for example. For example, slow release vehicles include macromers, poly(ethylene glycol), hyaluronic acid, poly(vinylpyrrolidone), or a hydrogel. By way of further example, the compositions may also include preserving agents, solubilising agents, stabilising agents, wetting agents, emulsifying agents, sweetening agents, colouring agents, flavouring agents, coating agents, buffers and the like. Those of skill in the art to which the invention relates can identify further additives that may be desirable for a particular purpose.

Hemichannel blockers may be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing hemichannel blockers may be prepared by known methods, including, for example, those described in: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy. Slow release delivery using PGLA nano- or microparticles, or in situ ion activated gelling systems may also be used, for example.

Additionally, it is contemplated that a hemichannel blocker pharmaceutical composition for use in accordance with the invention may be formulated with additional active ingredients or agents which may be of therapeutic or other benefit to a subject in particular instances. Persons of ordinary skill in the art to which the invention relates will appreciate suitable additional active ingredients having regard to the description of the invention herein and nature of the disorder to be treated.

The compositions may be formulated in accordance with standard techniques as may be found in such standard references as Gennaro A R: Remington: The Science and Practice of Pharmacy, 20^{th} ed., Lippincott, Williams & Wilkins, 2000, for example. However, by way of further example, the information provided in US2013/0281524 or U.S. Pat. No. 5,948,811 may be used.

In certain embodiments, the invention provides a combination product comprising, consisting essentially of, or consisting of (a) a hemichannel blockers and (b) one or more additional active agents, wherein the components (a) and (b) are adapted for administration simultaneously or sequentially.

In a particular embodiment of the invention, a combination product in accordance with the invention is used in a manner such that at least one of the components is administered while the other component is still having an effect on the subject being treated.

Any container suitable for storing and/or administering a pharmaceutical composition may be used for a hemichannel blocker product for use in a method of the invention.

The hemichannel blocker(s), for example, connexin 43 hemichannel blocker(s) may, in some aspects, be formulated to provide controlled and/or compartmentalized release to the site of administration. In some aspects of this invention, the formulations may be immediate, or extended or sustained release dosage forms. In some aspects, the dosage forms may comprise both an immediate release dosage form, in combination with an extended and/or sustained release dosage form. In some aspects both immediate and sustained and/or extended release of hemichannel blocker(s) can be obtained by combining a modified or unmodified peptide or peptidomimetic, for example, or other hemichannel blocker(s), in an immediate release form. In some aspects of this invention the hemichannel blockers are, for example, connexin 43 blockers or other hemichannel blockers of this disclosure. In some aspects of this invention, the dosage forms may be implants, for example, biodegradable or nonbiodegradable implants.

In some aspects of this invention, the hemichannel blocker, e.g., a connexin 43 hemichannel blocker, may be formulated for compartmentalized release of the blocker, for example, by adjusting the size or coating of the particles. For example, in some aspects, particle formulations of the hemichannel blocker, e.g., a connexin 43 blocker, can be administered for use in the methods of this invention. Drug delivery systems comprising particles may comprise, in some aspects, nanoparticles having a mean diameter of less than 1,000 nm, for example, 1-1000 nm, and/or microparticles having a mean diameter between 1 to 1,000 μm. The nanoparticles or microparticles may be, for example, nanospheres or microspheres, or encapsulated nanocapsules and microcapsules, in which the hemichannel blocker is encapsulated in a polymeric coating. The particle formulations may also comprise liposomes. In some aspects the hemichannel blocker is can include or exclude a blocker of a connexin 45, Cx26, Cx30, Cx31.1, Cx36, Cx37, Cx40, Cx50, or Cx57 hemichannel or any other connexin hemichannel in blood vessels. Preferred connexin targets are Cx36, Cx37, Cx43 and Cx 45 hemichannels. Especially preferred targets are Cx43 hemichannels.

The invention comprises methods for modulating the function of a hemichannel for the treatment of various disorders. Methods of the invention comprise administering a hemichannel blocker, alone or in a combination with one or more other agents or therapies as desired.

In another embodiment, hemichannel blockers, e.g., compounds of Formula I, for example Xiflam, compounds of Formula II, or peptide or peptidomimetic hemichannel blockers, may be administered systemically, such as by intravenous, intra-arterial or intraperitoneal administration, such that the final circulating concentration is from approximately 0.001 to approximately 150 micromolar, or higher up to 200, 300, 400, 500, 600, 700, 800, 900 or 1000 micromolar. The final circulating concentration can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, or 150 micromolar, or any concentration between any of the two recited numbers, or higher as described above and any concentration within the ranges noted. As mentioned herein, the invention also comprises combination therapies in which one or more additional active agent is also administered to a subject. Skilled persons will appreciate desirable dosages for the one or more active agent having regard to the nature of that agent and the principles discussed herein before. Preferred final circulating concentrations of active hemichannel modulators, or concentrations of hemichannel modulators at or about connexin hemichannel targets, e.g., tonabersat, hemichannel modulator compounds of Formula I, the hemichannel modulator compounds of Formula II, peptidomimetics (e.g., Peptide5), etc., range from 10-250 micromolar, 10-100 micromolar, 10-75 micromolar, 10-50 micromolar, 10-35 micromolar, 10-30 micromolar and 10-25 micromolar, and include 25 micromolar.

Administration of a hemichannel blocker, and optionally one or more other active agents, may occur at any time during the progression of a disorder, or prior to or after the development of a disorder or one or more symptom of a disorder. In one embodiment, a hemichannel blocker is administered periodically for an extended period to assist with ongoing management of symptoms. In another embodiment, a hemichannel blocker is administered periodically for an extended period or life-long to prevent or delay the development of a disorder.

In some embodiments, the hemichannel blockers, for example, a connexin 43 hemichannel blocker, can be administered as a pharmaceutical composition comprising one or a plurality of particles. In some aspects the pharmaceutical composition may be, for example, an immediate release formulation or a controlled release formulation, for example, a delayed release particle. In other aspects, hemichannel blockers can be formulated in a particulate formulation one or a plurality of particles for selective delivery to a region to be treated. In some embodiments, the particle can be, for example, a nanoparticle, a nanosphere, a nanocapsule, a liposome, a polymeric micelle, or a dendrimer. In some embodiments, the particle can be a microparticle. The nanoparticle or microparticle can comprise a biodegradable polymer. In other embodiments, the hemichannel blocker is prepared or administered as an implant, or matrix, or is formulated to provide compartmentalized release to the site of administration.

In some embodiments the formulated hemichannel blocker is a connexin 37 or connexin 40 or connexin 43 or connexin 45 hemichannel blocker. Connexin 37 or connexin 40 or connexin 43 blockers are preferred. Most preferred are connexin 43 hemichannel blockers. As used herein, “matrix” includes for example, matrices such as polymeric matrices, biodegradable or non-biodegradable matrices, and other carriers useful for making implants or applied structures for delivering the hemichannel blockers. Implants include reservoir implants and biodegradable matrix implants.

In some embodiments, a hemichannel blocker, e.g. a connexin 43 and hemichannel blocker, for example, is administered to the subject, providing therapeutically effective amounts of the connexin 43 hemichannel blocker using a microneedle, microneedle array, needle, or implant may be used for administration of the hemichannel blocker(s). In some embodiments, a microneedle may be used to administer a hemichannel blocker. In some embodiments, the penetration of the microneedle may be controlled to a desired depth within a tissue or organ or organ compartment. In some embodiments, the microneedle may also be coated with the a hemichannel blocker, alone or with other drug agents. In some aspects the volume of hemichannel blocker and/or drug agent administered by microneedle may be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 295, or 300 μl, or any range of volume between any two of the recited numbers or any volume between any two recited numbers. Any suitable formulation of this invention may be administered by microneedle injection, including, for example, nanoparticle or microparticle formulations, or other formulations injectable by microneedle.

### Articles of Manufacture/Kits of Combinations of Connexin Hemichannel Blockers

In another embodiment of the invention, an article of manufacture, or “kit”, containing materials useful for treating the diseases and disorders described above is provided. The kit comprises a container comprising, consisting essentially of, or consisting of connexin hemichannel blocker. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The container holds a hemichannel blocker, or a formulation thereof, which is effective for treating the condition and may have a sterile access port (e.g., the container may be an intravenous solution bag or a vial having a stopper pierceable by a hypodermic injection needle). The label or package insert indicates that the composition is used for treating the condition of choice, such any of the diseases, disorders and/or conditions described or referenced herein. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the hemichannel blocker to a patient in need thereof.

Articles of manufacturer are also provided, comprising, consisting essentially of, or consisting of a vessel containing a hemichannel blocker compound, composition or formulation and instructions for use for the treatment of a subject. For example, in another aspect, the invention includes an article of manufacture comprising, consisting essentially of, or consisting of a vessel containing a therapeutically effective amount of one or more connexin hemichannel blocker peptides or peptidomimetics and/or other hemichannel blocking agents, including small molecules, together with instructions for use, including use for the treatment of a subject.

In some aspects, the article of manufacture may comprise a matrix that comprises one or more connexin hemichannel blocker peptides or peptidomimetics or another hemichannel blocker, such as a small molecule hemichannel blocker, alone or in combination.

### Doses, Amounts and Concentrations

As will be appreciated, the dose of hemichannel blocker administered, the period of administration, and the general administration regime may differ between subjects depending on such variables as the target site to which it is to be delivered, the severity of any symptoms of a subject to be treated, the type of disorder to be treated, size of unit dosage, the mode of administration chosen, and the age, sex and/or general health of a subject and other factors known to those of ordinary skill in the art.

Examples of effective doses that may be used for the treatment of the diseases, disorders or conditions referenced herein are described. In some aspects, the therapeutically effective amount of the hemichannel blocker, for example a connexin 43 hemichannel blocker, is a concentration of about 0.001 to about 1.0 microgram/ml, or from about 0.001 to about 0.01 mg/ml, or from about 0.1 mg/mL to about 100 mg/mL, or more, or any range between any two of the recited dosages or any dose between any two recited numbers. The dose can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml or any range between any two of the recited dosages or any dose between any two recited numbers. In some embodiments, the therapeutically effective amount of the hemichannel blocker is present at a concentration ranging from about 0.5 to about 50 mg/mL. In some embodiments, the hemichannel blocker is present at a concentration ranging from about 0.3 to about 30 mg/mL. In some embodiments, the hemichannel blocker is present at a concentration ranging from about 0.1 or 1.0 to about 10 mg/mL. In some embodiments, the hemichannel blocker is present at a concentration ranging from about 0.1 or 1.0 to about 0.3 or 3.0 mg/mL. In some embodiments, the hemichannel blocker is present at a concentration of about 3.0 mg/mL.

In some aspects, the hemichannel blocker may be administered at a therapeutically effective dose between about 0.001 to about 100 mg/kg, between about 0.001 to about 0.01 mg/kg, between about 0.01 to about 0.1 mg/kg, between 0.1 to about 1 mg/kg, between about 1 to about 10 mg/kg, or between about 10 to about 100 mg/kg, or any range between any two recited dosages or any dose between any two recited dosages. In some aspects, the dose can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml or any range between any two of the recited dosages or any dose between any two recited numbers.

It should be appreciated that administration may include a single daily dose, administration of a number of discrete divided doses, or continuous administration, as may be appropriate. By way of example, unit doses may be administered once or more than once per day, for example 1, 2, 3, 4, 5 or 6 times a day to achieve a desired total daily dose. By way of example, a unit dose of a hemichannel blocker may be administered in a single daily dose or a number of discrete doses, or continuously to achieve a daily dose of approximately 0.1 to 10 mg, 10 to 100 mg, 100 to 1000 mg, 1000 to 2000 mg, or 2000 mg to 5000 mg, 0.1 to approximately 2000 mg, approximately 0.1 to approximately 1000 mg, approximately 1 to approximately 500 mg, approximately 1 to approximately 200 mg, approximately 1 to approximately 100 mg, approximately 1 to approximately 50 mg, or approximately 1 to approximately 25 mg, or any range between any two recited dosages or any dose between any two recited dosages.

By way of further example, a unit dose of a hemichannel blocker may be administered once or more than once a day (for example 1, 2, 3, 4, 5 or 6, typically 1 to 4 times a day), such that the total daily dose is in the range (for a 70 kg adult) of approximately 1 to approximately 1000 mg, for example approximately 1 to approximately 500 mg, or 500 mg to 1000 mg, 1000 to 2000 mg, or 2000 mg to 5000 mg, or any range between any two recited dosages or any dose between any two recited dosages. For example, a hemichannel blocker, such as Peptagon, and/or an analogue thereof, compounds of Formula I, for example Xiflam, and analogs of any of the foregoing compounds, may be administered to a subject at a dose range of approximately 0.01 to approximately 15 mg/kg/day, for example approximately 0.1 to approximately 6 mg/kg/day, for example approximately 1 to approximately 6 mg/kg/day, for example, 6 mg/kg/day to 100 mg/kg/day or any range between any two recited dosages or any dose between any two recited dosages. In one embodiment, Xiflam may be administered orally once a day at a dose of approximately 2 mg to approximately 40 mg.

In one embodiment, the dose of a hemichannel blocker is approximately 0.001 micromolar to 0.1 micromolar, 0.1 micromolar and up to approximately 200 micromolar at the site of action, or higher, within the circulation to achieve those concentrations at the site of action. By way of example, the dose may be (but not limited to) a final circulating concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micromolar, or any range between any two recited concentrations, or any concentration between any two recited numbers. Further examples of doses expected to block hemichannels but not to uncouple gap junctions are described in O'Carroll et al, 2008, herein incorporated by reference. In some embodiments, Xiflam may be used at a lower dose, for example, 0.001 to 20 micromolar. A low dose can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, or 20 micromolar.

In one embodiment, the dose of a hemichannel blocker, such as Peptagon and/or an analogue thereof, is approximately 0.001 micromolar and up to approximately 200 micromolar, or 200 to 2000 or 5000 micromolar at the site of action, or higher within the circulation to achieve those concentrations at the site of action. By way of example, the dose may be (but not limited to) a final circulating concentration of about 1, 5, 10, 20, 50, 100, 200, 250, 500, 1000, 2000, 3000, 4000, or 5000 micromolar, or any range between any two recited dosages or any dose between any two recited dosages. Doses of Peptagon effective to block hemichannels but not to uncouple gap junctions are discussed in O'Carroll et al, 2008.

In some embodiments, Xiflam may be used at a lower dose, for example, 1 to 20 micromolar, 1 to 50 micromolar, 20 to 30, 30 to 40 or 40 to 50 micromolar. A low dose can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, or 20 micromolar.

In some embodiments, a suitable therapeutically effective dose of a hemichannel blocker thereof, may be at least about 1.0 mg/mL of the hemichannel blocker. In some embodiments, the therapeutically effective dose of the hemichannel blocker may be from about 0.001 mg/mL to 0.01 mg/mL, from about 0.01 mg/mL to about 0.1 mg/mL, or from about 0.1 mg/mL to about 100 mg/mL. In some embodiments, the suitable therapeutically effective dose of hemichannel blocker may be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 52.5, 55.0, 57.5, 60.0, 62.5, 65.0, 67.5, 70.0, 72.5, 75.0, 77.5, 80.0, 82.5, 85.0, 87.5, 90.0, 92.5, 95.0, 97.5, or about 100.0 ug/mL, or any range or subrange between any two of the recited doses, or any dose falling within the range of about 0.1 to about 100 ug/mL. In some embodiments, the suitable therapeutically effective dose of a hemichannel blocker may be about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 52.5, 55.0, 57.5, 60.0, 62.5, 65.0, 67.5, 70.0, 72.5, 75.0, 77.5, 80.0, 82.5, 85.0, 87.5, 90.0, 92.5, 95.0, 97.5, or about 100.0 mg/mL, or any range or subrange between any two of the recited doses, or any dose falling within the range of about 0.1 to about 100 mg/mL. In some embodiments, the hemichannel blocker, is present at a concentration ranging from about 0.5 to about 50 mg/mL. In other embodiments, the hemichannel blocker is present at a concentration ranging from about 0.3 to about 30 mg/mL. In other embodiments, the hemichannel blocker is present at a concentration ranging from about 0.1 or 1.0 to about 10 mg/mL. In other embodiments, the hemichannel blocker is present at a concentration ranging from about 0.1 or 1.0 to about 0.3 or 3.0 mg/mL. In other embodiments, a hemichannel blocker, such as a connexin 43 hemichannel blocker, and/or a connexin 45 hemichannel blocker is present at a concentration of about 3.0 mg/mL. In any of these aspects the hemichannel blocker, may be a connexin 43 or connexin 45 hemichannel blocker. When the hemichannel blocker is a modified or unmodified peptide or peptidomimetic, the dose may be decreased by 1-10, 25-50, 100-200, or 1000 fold.

In certain embodiments, the hemichannel blockers, for example, a connexin 43 hemichannel blocker, may be administered at about 0.001 micromolar (μM) or 0.05 μM to about 200 μM, or up to 300 μM or up to 1000 μM or up to 2000 μM or up to 3200 μM or more, for example up to about 10 mM, 20 mM, or 30 mM final concentration at the treatment site and/or adjacent to the treatment site, and any doses and dose ranges within these dose numbers. In one embodiment, the hemichannel blocker composition is applied at greater than about 1000 μM. Preferably, the hemichannel blocker composition is applied at about 1000 μM to about 10 mM final concentration, more preferably, the anti-connexin agent composition is applied at about 3 mM to about 10 mM final concentration, and more preferably, the hemichannel blocker composition is applied at about 1-3 mM to about 5-10 mM final concentration. The hemichannel blocker concentration can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 micromolar; or 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 millimolar, or any range between any two of the recited dosages or any dose between any two recited numbers.

Additionally, hemichannel blockers, for example, connexin 43 hemichannel blockers may be present in the formulation at about 1 μM to about 50 μM final concentration, and alternatively the connexin 43 hemichannel blocker, for example, is present at about 5 μM to about 20 μM final concentration, or at about 10 to about 15 μM final concentration. In certain other embodiments, the hemichannel blocker is present at about 10 μM final concentration. In yet another embodiment, the hemichannel blocker is present at about 1-15 μM final concentration. In other embodiments, the hemichannel blocker is present at about 20 μM, 30 μM, 40 μM, 50 μM, 60 μM, 70 μM, 80 μM, 90 μM, 100 μM, 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM, 2000 μM-3000 μM, 3000 μM-4000 μM, 4000 μM-5000 μM, 5000 μM-6000 μM, 6000 μM-7000 μM, 7000 μM-8000 μM, 8000 μM-9000 μM, 9000 μM-10,000 μM, 10,000 μM-11,000 μM, 11,000 μM-12,000 μM, 12,000 μM-13,000 μM, 13,000 μM-14,000 μM, 14,000 μM-15,000 μM, 15,000 μM-20,000 μM, 20,000 μM-30,000 μM, 30,000 μM-50,000 μM, or greater, or any range or subrange between any two of the recited doses, or any dose falling within the range of from about 20 μM to about 50,000 μM.

Still other dosage levels between about 1 nanogram (mg)/kg and about 1 mg/kg body weight per day of each of the hemichannel blockers described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one hemichannel blocker is used, the dosage of each hemichannel blocker need not be in the same range as the other. For example, the dosage of one connexin hemichannel blocker may be between about 0.01 mg to about 10 mg per kg body weight, and the dosage of another connexin hemichannel blocker may be between about 0.1 mg to about 1 mg per kg body weight, 0.1 to about 10, 0.1 to about 20, 0.1 to about 30, 0.1 to about 40, or between about 0.1 to about 50 mg per kg body weight. The dosage may also be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg body weight or any range or subrange between any two of the recited doses, or any dose falling within the range of from about 0.001 to about 100 mg per kg body weight.

As noted above, doses of a hemichannel blocker, for example, a connexin 37, 40 or 43 hemichannel blocker, may be administered in single or divided applications. The doses may be administered once, or application may be repeated. Typically, application will be repeated weekly, biweekly, or every 3 weeks, every month, or every 2, 3, 4, 5, 6, 7, 8, 9, 10, 11, 12, 13, 14, 15, 16, 17, 18, 19, 20, 21, 22, 23, or every 24 months or more as needed to prevent, slow, or treat any disease, disorder or condition described herein. Doses may also be applied every 12 hours to 7 days apart, or more. For example, doses may be applied 12 hours, or 1, 2, 3, 4, 5, 6, or 7 days apart, or at any time interval falling between any two of these times, or between 12 hours and 7 days. The connexin 43 hemichannel blocker, for example, may be administered for up to four, six, eight, ten, twelve, fourteen, sixteen, eighteen, twenty, twenty-two, twenty-four or twenty-six weeks. For some indications, more frequent dosing, may employed.

### Manufacture and Purity

Small molecule hemichannel blockers, including those of Formula I and II may be prepared as previously described. Methods of synthesizing antibodies and binding fragments as well as peptides and polypeptides, including peptidomimetics and peptide analogs can be performed using suitable methods. See e.g. Lihu Yang et al., Proc. Natl. Acad. Sci. U.S.A., 1; 95(18): 10836-10841 (Sep. 1 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

In some embodiments, the formulations of this invention are substantially pure. By substantially pure is meant that the formulations comprise less than about 10%, 5%, or 1%, and preferably less than about 0.1%, of any impurity. In some embodiments the total impurities, including metabolites of the connexin 43 modulating agent, will be not more than 1-15%. In some embodiments the total impurities, including metabolites of the connexin 43 modulating agent, will be not more than 2-12%. In some embodiments the total impurities, including metabolites of the connexin 43 modulating agent, will be not more than 3-11%. In other embodiments the total impurities, including metabolites of the connexin 43 modulating agent, will be not more than 4-10%.

### EXAMPLES

The work described in these Examples evaluated and demonstrated the positive effect of hemichannel blockers on BRB integrity, RPE integrity, tight junction integrity and ZO-1 internalization, and decreases in type IV collagen production.

### Example 1

### Methods

Cell Culture—Human adult retinal pigment epithelial cells (ARPE-19; American Type Culture Collection, USA) were cultured in Dulbecco's Modified Eagle Medium Nutrient Mixture F-12 (DMEM-F12; Thermofisher Scientific Inc., USA) supplemented with 10% foetal bovine serum (FBS; Invitrogen, USA) and a 1× antibiotics and antimycotics mixture (AA, 100× stock) at 37° C. in a humidified 5% CO₂ incubator. Cells were grown in T75 flasks and the medium was changed twice per week until confluent.

HG and/or cytokine challenge—At passage 6-12, cells were plated at 2.5×10⁵ cells/mL in 8-well chamber slides for immunohistochemical studies, 6-well plates for TEER and FITC-dextran studies or 96-well plates for the lactose dehydrogenase (LDH) and ATP release assay until confluent after which the culture medium was changed to treatments in serum-free DMEM-F12 containing 1×AA for 24 h. DR-like conditions were induced as previously described [23,20,21]. Briefly, cells were challenged with a combination of 32.5 mM HG and pro-inflammatory cytokines, tumour necrosis factor alpha (TNF-α; 10 ng/mL; Peprotech, USA) and interleukin-1 beta (IL-1β; 10 ng/mL; Peprotech, USA).

Application of treatments—Peptide5 (H-Val-Asp-Cys-Phe-Leu-Ser-Arg-Pro-Thr-Glu-Lys-Thr-OH (SEQ ID NO: 1); China Peptides, China) was administered at a concentration of 25 μM to cells at the same time as the combination of HG and pro-inflammatory cytokines [20]. For experiments assessing the effect of extracellular ATP, exogenous ATP (100 nM) was added to cells at the same time as injury and Peptide5 treatment.

Measurement of Trans-Epithelial Electrical Resistance (TEER)—Cells were seeded at 2.5×10⁵ cells/mL on polyester membranes in Transwell® 6-well plates (Corning Incorporated, USA) in growth medium and incubated for 72 h. The medium was then changed to serum free plating medium containing treatments. TEER measurements were obtained at 0, 24, 48, and 72 h following treatments using the EVOM2 (World Precision Instruments, USA) with an STX3 electrode. Net TEER values were calculated by subtracting the resistance in Transwell inserts without cells from the experimental values obtained from chamber containing cells. The net TEER was multiplied by the area of the insert to give the TEER in Ω·cm². TEER data was reported relative to basal conditions. The sample size was three readings per well, repeated three times in separate experiments.

Measurement of FITC-dextran paracellular permeability—The integrity of tight junctions between ARPE-19 cells was examined by measuring the movement of a 70,000 Da fluorescein isothiocynate (FITC)-dextran (D1820, Thermofisher Scientific Inc., USA) across a monolayer of cells. Following TEER measurements at 72 h, 1000 μL of spent medium in the inserts was replaced by 1000 μL FITC-dextran (10 μg/mL) and incubated for 1 h. Inserts were removed and samples were transferred to 96-well plates for quantification by spectrophotometry (excitation 490 nm and emission 520 nm). FITC-dextran permeability was expressed as a percentage relative to blank wells containing no cells and no treatments. The sample size was three readings per well, repeated three times in separate experiments.

ATP release assay—ATP released into the cell culture medium was measured as previously described [20] using the ATPLite Luminescence ATP Detection Kit (PerkinElmer, USA). ATP release was presented as a percentage of basal conditions. The sample size was six wells per group, repeated three times in separate experiments.

Lactate Dehydrogenase (LDH) assay—Cells were seeded at 2.5×10⁵ cells/mL in 96-well plates until confluent after which the culture medium was changed to treatments in serum-free DMEM-F12 containing 1×AA for 72 h. After 72 h of incubation in media containing treatments, 50 μL of culture medium was taken from each well to measure LDH release. The sample size was six wells per group, repeated three times in separate experiments. The amount of LDH released was assessed using an LDH assay kit as per manufacturer instructions (Sigma-Aldrich, USA). In brief, LDH reduces NAD to NAD+, which then converts a tetrazolium dye to soluble and coloured formazan. A Synergy 2 multi-mode plate reader (BioTek Instruments Inc., USA) was used to measure the absorbance of the formazan dye in the medium at 490 nm (OD490). LDH release (%) was calculated relative to basal conditions.

Immunohistochemistry—Cells were seeded at 2.5×10⁵ cells/mL in 8-well chamber slides for immunohistochemistry experiments. After 72 h of incubation in treatment media, cells were fixed with 4% paraformaldehyde for 10 min and permeabilised with 0.1% Triton X-100 in phosphate buffer saline (PBS) for 10 min. Cells were then incubated with either rabbit anti-ZO-1 (1:400; Invitrogen, USA), rabbit anti-connexin43 (1:2000; Sigma-Aldrich, USA), or mouse anti-collagen IV (1:1000; Sigma-Aldrich, USA) at 4° C. overnight followed by washing in PBS three times for 15 min. Goat anti-rabbit Alexa-488 (1:500; Invitrogen, USA) or goat anti-mouse Cy3 (1:500; Jackson Immuno Research, USA) secondary antibodies were applied and incubated at room temperature for 3 h. Secondary-only controls revealed no non-specific labelling. Cell nuclei were stained with DAPI (1:1000; Sigma-Aldrich, USA) and slides were mounted using Citifluor™ anti-fade reagent. Labelling was repeated three times in separate experiments.

Image analysis and quantification of collagen IV immunolabelling—Images were taken on an Olympus FV1000 confocal laser scanning microscope (Olympus, Japan). Images were processed using the Olympus FV-10 ASW viewer and version 1.46r of the ImageJ software (National Institute of Health, USA). Both ZO-1 and connexin43 expression were qualitatively assessed for changes in localisation. Collagen IV labelling was quantified from four images taken per well and the experiment was repeated three times. Using the ImageJ software, each image was split into RGB channels with collagen IV in the red channel. The image was converted into an 8-bit binary image and an equal threshold was applied to every image to reduce background and avoid bias. The total area covered by collagen IV was then quantified using the ‘measure’ feature in ImageJ. Collagen IV results were expressed as a percentage of untreated (basal) cells.

Statistical analysis—Data are presented as arithmetic means±S.D. Statistical comparisons between groups were performed using one-way ANOVA or two-way ANOVA with Dunnet's multiple correction's test using GraphPad Prism 6. The specific statistical method used for each data set is provided in the respective figure legend. Adjusted p<0.05 was considered to indicate statistically significant differences.

### Example 2

### Treatment with Hemichannel Blockers Prevented a Decrease in TEER and Protected Against a Corresponding Increase in Paracellular Permeability

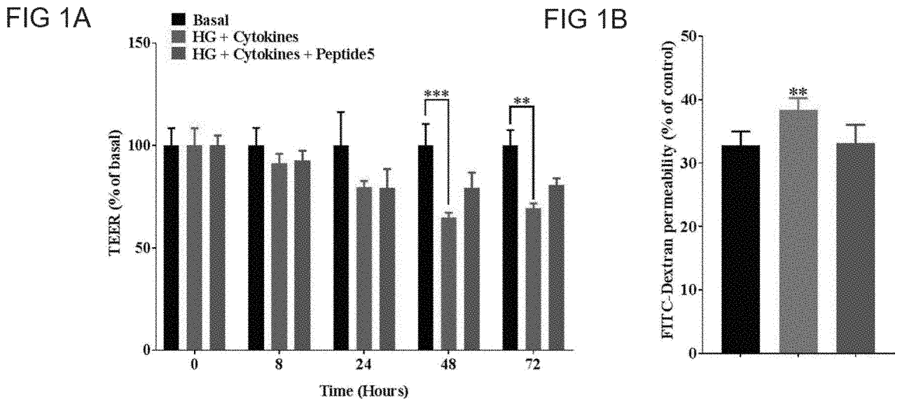

The combination of HG and cytokines resulted in a decrease in TEER at 48 h (p=0.0007) and 72 h (p=0.0030) compared to basal conditions (FIG. 1A). This was accompanied by a significant increase in FITC-dextran permeability (p=0.0016) at 72 h following the addition of HG and inflammatory cytokines (38.42±1.84%) relative to basal conditions (32.77±2.23%) (FIG. 1B). Hemichannel block with Peptide5 treatment reduced the decrease in TEER at both time-points such that there was no statistically significant difference between Peptide5-treated and basal cells at both 48 h (p=0.2238) and 72 h (p=0.3778). Similarly, hemichannel block using a model blocker, Peptide5 (33.2±2.87%), protected against the increase in FITC-dextran permeability at 72 h.

### Example 3

### Hemichannel Blocker Treatment Prevented Collagen IV Upregulation

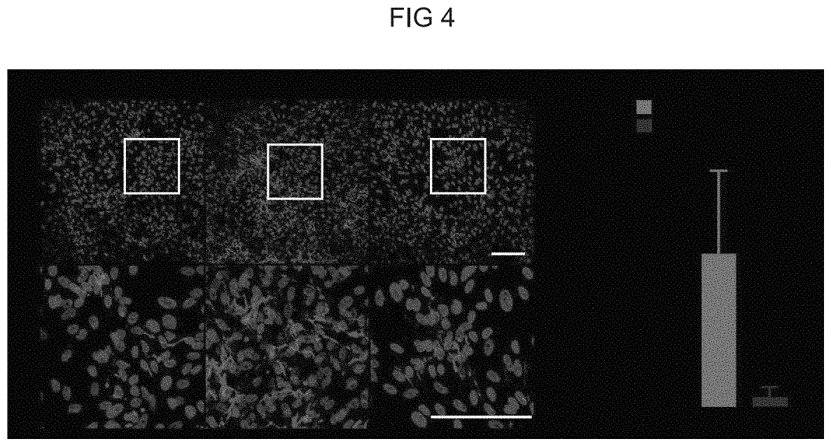

ARPE-19 cells deposited low levels of collagen IV under basal conditions (FIG. 4). With the addition of HG and inflammatory cytokines, there was an increase in collagen IV deposition (618.5±332.3%; p=0.0180) compared to basal cells (31.25±36.06%). Hemichannel blocker treatment with Peptide5 prevented collagen IV upregulation with no statistically significant difference between Peptide5-treated (40.32±43.16%) and basal cells (p=0.9976).

### Example 4

### Connexin Hemichannel Block Protected Against Loss of Connexin43 Localisation at the Cell Membrane

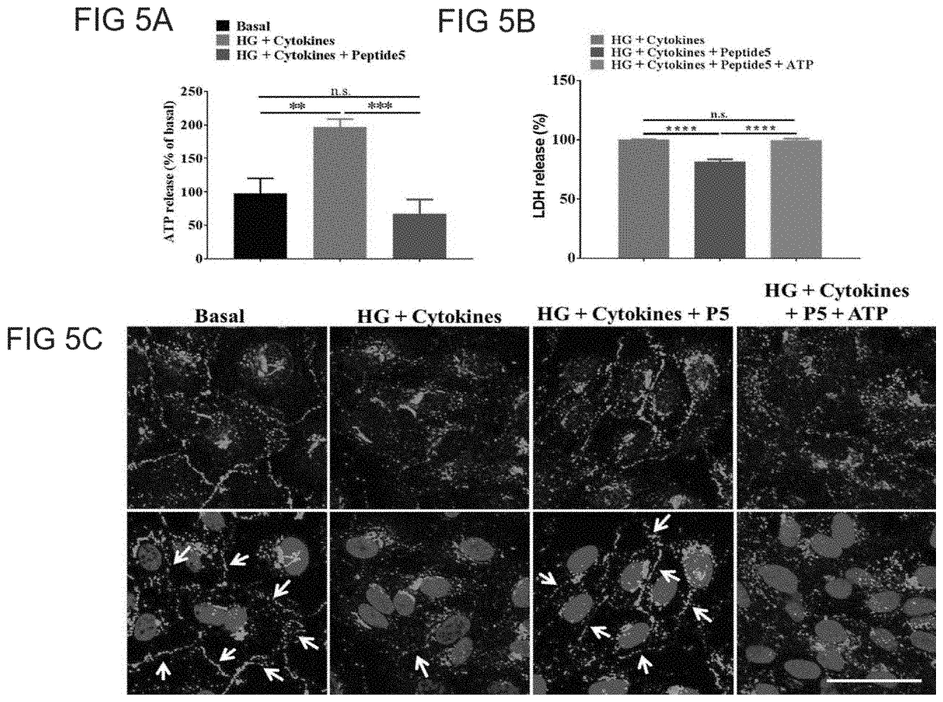

Basally, connexin43 protein was localised at cell membranes in plaques that could be immunohistochemically labelled (FIG. 5C). With the addition of HG and inflammatory cytokines, there was a loss of connexin43 plaque labelling at the cell membrane and an increase in intracellular localisation of the protein. Hemichannel blocker treatment with Peptide5 protected against the redistribution of connexin43 localisation away from the cell membrane and into the cytoplasm.

### Example 5

### Peptides Protected Against HG and Cytokine-Induced ATP Release

HG and cytokines (196.5±12.15%) induced increased ATP release relative to basal conditions (98.25±21.91%; p=0.0014) (FIG. 5A). Hemichannel blocker treatment with Peptide5 (66.67±21.91%) prevented HG and cytokine-mediated ATP release (p=0.0003) such that there was no statistically significant difference between basal and Peptide5-treated cells.

### Example 6

### Peptides Protected Against HG and Cytokine-Induced Cell Injury in an ATP-Dependent Manner

To confirm that the protective effect was based on hemichannel blocker treatment, exogenous ATP was added to the cell culture medium in the presence of HG and inflammatory cytokines as well as Peptide5. As previously shown, LDH release increases and there is an increase in connexin43 internalization to the cell cytoplasm with the addition of HG and cytokines; however, connexin43 is kept in its normal pattern and LDH release remains low in the presence of Peptide5. Exogenously added ATP, however, reversed the protection conferred by the Peptide5 hemichannel blocker as measured by a once-again increased LDH release (FIG. 5B) and a change in connexin43 gap junction localisation (FIG. 5C).

### Discussion

TEER and FITC-dextran permeability were used as markers to assess the barrier properties of ARPE-19 cells following injury with a combination of HG and pro-inflammatory cytokines without and with treatment using a hemichannel blocker, in this case the Peptide5 hemichannel blocker. Results showed that blocking connexin43 hemichannels was able to prevent HG and cytokine-mediated decrease in TEER and the increase in FITC-dextran permeability, supporting the idea that connexin43 hemichannels can effectively mediate RPE disruption and BRB disruption in, for example, DME, through RPE and BRB integrity/function modulation. Importantly, it was also demonstrated that blocking connexin43 hemichannels protected tight junction integrity and maintained ZO-1 localisation at the cell membrane.

Increased secretion of extracellular matrix components has also been reported as a feature of stressed RPE cells. Trudeau and colleagues found that a combination of HG and IL-1β increases collagen IV gene and protein expression by RPE cells in vitro. Trudeau K, et al. (2011) Fenofibric acid reduces fibronectin and collagen type IV overexpression in human retinal pigment epithelial cells grown in conditions mimicking the diabetic milieu: functional implications in retinal permeability. Invest Ophthalmol Vis Sci 52 (9):6348-6354. This is in line with the results herein showing that collagen IV expression by ARPE-19 cells increased in response to HG and cytokines. Furthermore, higher collagen IV expression has been found in the basement membrane of donors with confirmed DR diagnosis compared to normal donors (Roy S, et al. (1994) Increased expression of basement membrane collagen in human diabetic retinopathy. J Clin Invest 93 (1):438-442), further demonstrating the importance of the discovery herein that hemichannel blocker treatment prevented collagen IV upregulation. Previous studies have shown that reducing collagen IV upregulation prevents basement membrane thickening which can in turn prevent RPE barrier breakdown. Id. Therefore, blocking hemichannel, for example with connexin 43 hemichannel blockers, can guard RPE integrity not just through protection of tight junctions but by helping to maintain cellular homeostasis. This is also supported by the findings herein on LDH release from ARPE-19 cells. While LDH release levels shown indicate a loss of cell membrane integrity as opposed to cell death, hemichannel block was able to protect against an increase in LDH release, which also supports the conception that connexin43 hemichannel block will help maintain cell membrane integrity. Taken together, the ZO-1, collagen IV and LDH results support the idea that blocking the ATP-dependent inflammasome activation induced by pathological and unregulated connexin43 hemichannel opening results in maintenance of tight junction, basement membrane and cell membrane structure.

In line with these results is the discovery herein that gap junctional connexin43 localization was disrupted following injury with a combination of HG and cytokines. It has previously been reported that connexin43 protein expression increases in the retina in a mouse model of DR and in donors with confirmed DR diagnosis. Mugisho O O, et al. (2017) Immunohistochemical Characterization of Connexin43 Expression in a Mouse Model of Diabetic Retinopathy and in Human Donor Retinas. Int J Mol Sci 18 (12):2567. The present study supports the idea that connexin43 gap junction plaques can also be redistributed in the plasma membrane or become internalized in disease. As cell-cell communication via gap junctions are required for normal cell functioning, loss of gap junctions at the cell membrane suggests a pathological state resulting in loss of cellular homeostasis. Connexin43 hemichannel block maintained normal gap junctional connexin43 distribution at the cell membrane, and therefore cellular homeostasis. See Eugenin E A, et al. (2012) The role of gap junction channels during physiologic and pathologic conditions of the human central nervous system. J Neuroimmune Pharmacol 7 (3):499-518.

Previous studies have also reported that extracellular ATP is a key signalling molecule that initiates the NLRP3 inflammasome pathway. The above experiments evaluated the role of ATP in connexin43 hemichannel-mediated RPE barrier breakdown. Results showed that LDH release was increased and connexin43 gap junctions were disrupted in the presence of exogenously added ATP, even with the connexin43 hemichannel blocker, Peptide5, also present in the medium. The these finding indicate that the connexin43 hemichannel role in RPE barrier permeability may also involve mediation of ATP release.

In conclusion, while previous studies have suggested that a loss of RPE barrier integrity is primarily a ZO-1 (tight junction) related defect that is independent of connexin43 activity, Obert E, et al. (2017) Targeting the tight junction protein, zonula occludens-1, with the connexin43 mimetic peptide, alphaCT1, reduces VEGF-dependent RPE pathophysiology. J Mol Med (Berl) 95 (5):535-552, the present study shows that the loss of RPE and BRB integrity and function that occurs, as well as loss of tight junction integrity and function, for example, is initiated primarily by pathological opening of connexin hemichannels, and connexin43 hemichannels in particular. Connexin43 hemichannel opening leads to ATP release which in turn activates the NLRP3 inflammasome pathway. It was here discovered that this results in a loss of ZO-1 and gap junctional connexin43 localisation at the cell membrane which contributes to loss of barrier integrity and function and cellular homeostasis, reflected by collagen IV expression and LDH release. These results further support the concept that targeting hemichannels can protect against the loss of RPE and BRB integrity, as well as the loss of tight junction integrity and increase in collage IV production, that occur in various diseases, disorders and conditions. As noted, preferred connexin hemichannel targets include not only Cx 43 hemichannels, but the Cx36, Cx 37 and Cx45 hemichannels that are also found in the retina.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, and in embodiments or examples of the present invention, any of the terms “comprising”, “consisting essentially of”, and “consisting of” may be replaced with either of the other two terms in the specification. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method for modulating blood retinal barrier (BRB) integrity in a subject, comprising administering an effective amount of a hemichannel blocker to said subject.

2. A method for modulating retinal pigment epithelial (RPE) integrity in a subject, comprising administering an effective amount of a hemichannel blocker to said subject.

3. A method for modulating tight junction integrity in a subject, comprising administering an effective amount of a hemichannel blocker to said subject.

4. A method for modulating type IV collagen production in a subject, comprising administering an effective amount of a hemichannel blocker to said subject.

5. A method for reducing gap junction plaque internalization in cells in a subject, comprising administering an effective amount of a hemichannel blocker to said subject.

6. The method of any of claim 1-4 or 5, wherein the hemichannel blocker is a connexin 43 hemichannel blocker.

7. The method of any of claim 1-4 or 5, wherein the hemichannel blocker is a small molecule hemichannel blocker according to Formula I.

8. The method of claim 7, wherein the small molecule hemichannel blocker is N-[(3S,4S)-6-acetyl-3-hydroxy-2,2-dimethyl-3,4-dihydrochromen-4-yl]-3-chloro-4-fluorobenzamide (Xiflam).

9. The method of claim 7, wherein the small molecule hemichannel blocker is a Xiflam prodrug.

10. The method of any of claim 1-4 or 5, wherein the hemichannel blocker is a peptidomimetic connexin 43 hemichannel blocker.

11. The method of claim 10, wherein the peptidomimetic connexin 43 hemichannel blocker is Peptide5.

12. The method of claim 1, wherein said hemichannel blocker is administered to achieve a final circulating concentration of the hemichannel blocker ranging from about 10 to about 250 micromolar.

13. The method of claim 1, wherein said hemichannel blocker is administered by injection.

14. The method of claim 1, wherein said hemichannel blocker is administered orally.

15. The method of claim 1, wherein the hemichannel blocker is administered administered PRN or on a predetermined schedule or both.

16. The method of claim 1, wherein the subject is a human.


SEQUENCE LISTING (175 sequences)

SEQ ID NO: 1 - protein, 12 aa, Artificial Sequence
  Description of Artificial Sequence: Synthetic peptide
VDCFLSRPTEKT

SEQ ID NO: 2 - protein, 7 aa, Artificial Sequence
  Description of Artificial Sequence: Synthetic peptide
SRPTEKT

SEQ ID NO: 3 - protein, 12 aa, Artificial Sequence
  Description of Artificial Sequence: Synthetic peptide
ADCFLSRPTEKT

SEQ ID NO: 4 - protein, 12 aa, Artificial Sequence
  Description of Artificial Sequence: Synthetic peptide
VACFLSRPTEKT

SEQ ID NO: 5 - protein, 12 aa, Artificial Sequence
  Description of Artificial Sequence: Synthetic peptide
VDCFLSRPTAKT

SEQ ID NO: 6 - protein, 12 aa, Artificial Sequence
  Description of Artificial Sequence: Synthetic peptide
VDCFLSRPTEAT

SEQ ID NO: 7 - protein, 10 aa, Artificial Sequence
  Description of Artificial Sequence: Synthetic peptide
CFLSRPTEKT

SEQ ID NO: 8 - protein, 8 aa, Artificial Sequence
  Description of Artificial Sequence: Synthetic peptide
LSRPTEKT

SEQ ID NO: 9 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 10 - protein, 35 aa, Artificial Sequence
  Description of Artificial Sequence: Synthetic polypeptide
ESAWGDEQSAFRCNTQQPGCENVCYDKSFPISHVR

SEQ ID NO: 11 - protein, 36 aa, Artificial Sequence
  Description of Artificial Sequence: Synthetic polypeptide
GESIYYDEQSKFVCNTEQPGCENVCYDAFAPLSHVR

SEQ ID NO: 12 - protein, 38 aa, Artificial Sequence
  Description of Artificial Sequence: Synthetic polypeptide
LLIQWYIYGFSLSAVYTCKRDPCPHQVDCFLSRPTEKT

SEQ ID NO: 13 - protein, 38 aa, Artificial Sequence
  Description of Artificial Sequence: Synthetic polypeptide
LIGQYFLYGFQVHPFYVCSRLPCHPKIDCFISRPTEKT

SEQ ID NO: 14 - protein, 16 aa, Artificial Sequence
  Description of Artificial Sequence: Synthetic peptide
VDCFLSRPTEKTIFII

SEQ ID NO: 15 - protein, 13 aa, Artificial Sequence
  Description of Artificial Sequence: Synthetic peptide
DCFISRPTEKTIF

SEQ ID NO: 16 - protein, 11 aa, Artificial Sequence
  Description of Artificial Sequence: Synthetic peptide
SRPTEKTIFII

SEQ ID NO: 17 - protein, 42 aa, Artificial Sequence
  Description of Artificial Sequence: Synthetic polypeptide
LLIQWYIYGFSLSAVYTCKRDPCPHQVDCFLSRPTEKTIFII

SEQ ID NO: 18 - protein, 42 aa, Artificial Sequence
  Description of Artificial Sequence: Synthetic polypeptide
LIGQYFLYGFQVHPFYVCSRLPCHPKIDCFISRPTEKTIFLL

SEQ ID NO: 19 - protein, 9 aa, Artificial Sequence
  Description of Artificial Sequence: Synthetic peptide
KQIEIKKFK

SEQ ID NO: 20 - protein, 26 aa, Artificial Sequence
  Description of Artificial Sequence: Synthetic peptide
DGVNVEMHLKQIEIKKFKYGIEEHGK

SEQ ID NO: 21 - protein, 26 aa, Artificial Sequence
  Description of Artificial Sequence: Synthetic peptide
DGVNVEMHLKQIEIKKFKYGIEEQGK

SEQ ID NO: 22 - protein, 20 aa, Artificial Sequence
  Description of Artificial Sequence: Synthetic peptide
YGRKKRRQRRRKQIEIKKFK

SEQ ID NO: 23 - protein, 17 aa, Artificial Sequence
  Description of Artificial Sequence: Synthetic peptide
CSSPTAPLSPMSPPGYK

SEQ ID NO: 24 - protein, 11 aa, Artificial Sequence
  Description of Artificial Sequence: Synthetic peptide
PTAPLSPMSPP

SEQ ID NO: 25 - protein, 7 aa, Artificial Sequence
  Description of Artificial Sequence: Synthetic peptide
RPRDDEI

SEQ ID NO: 26 - protein, 9 aa, Artificial Sequence
  Description of Artificial Sequence: Synthetic peptide
SRPRDDLEI

SEQ ID NO: 27 - protein, 19 aa, Artificial Sequence
  Description of Artificial Sequence: Synthetic peptide
YGRKKRRQRRRSRPRDDEI

SEQ ID NO: 28 - protein, 18 aa, Artificial Sequence
  Description of Artificial Sequence: Synthetic peptide
YGRKKRRQRRRRPRDDEI

SEQ ID NO: 29 - protein, 6 aa, Artificial Sequence
  Description of Artificial Sequence: Synthetic peptide
  MOD_RES (4)..(4): 4Hyp
GAGXPY

SEQ ID NO: 30 - protein, 20 aa, Artificial Sequence
  Description of Artificial Sequence: Synthetic peptide
PSSRASSRASSRPRPDDLEI

SEQ ID NO: 31 - protein, 9 aa, Artificial Sequence
  Description of Artificial Sequence: Synthetic peptide
RPRPDDLEI

SEQ ID NO: 32 - protein, 9 aa, Artificial Sequence
  Description of Artificial Sequence: Synthetic peptide
RPRPDDLEV

SEQ ID NO: 33 - protein, 9 aa, Artificial Sequence
  Description of Artificial Sequence: Synthetic peptide
RPRPDDVPV

SEQ ID NO: 34 - protein, 9 aa, Artificial Sequence
  Description of Artificial Sequence: Synthetic peptide
KARSDDLSV

SEQ ID NO: 35 - protein, 40 aa, Artificial Sequence
  Description of Artificial Sequence: Synthetic polypeptide
QKPEVPNGVSPGHRLPHGYHSDKRRLSKASSKARSDDLSV

SEQ ID NO: 36 - protein, 36 aa, Artificial Sequence
  Description of Artificial Sequence: Synthetic polypeptide
RQPKIWFPNRRKPWKKPSSRASSRASSRPRPDDLEI

SEQ ID NO: 37 - protein, 36 aa, Artificial Sequence; identical to SEQ ID NO: 36
  Description of Artificial Sequence: Synthetic polypeptide

SEQ ID NO: 38 - protein, 25 aa, Artificial Sequence
  Description of Artificial Sequence: Synthetic peptide
RQPKIWFPNRRKPWKKRPRPDDLEI

SEQ ID NO: 39 - protein, 25 aa, Artificial Sequence
  Description of Artificial Sequence: Synthetic peptide
RQPKIWFPNRRKPWKKRPRPDDLEV

SEQ ID NO: 40 - protein, 25 aa, Artificial Sequence
  Description of Artificial Sequence: Synthetic peptide
RQPKIWFPNRRKPWKKRPRPDDVPV

SEQ ID NO: 41 - protein, 25 aa, Artificial Sequence
  Description of Artificial Sequence: Synthetic peptide
RQPKIWFPNRRKPWKKKARSDDLSV

SEQ ID NO: 42 - protein, 9 aa, Artificial Sequence
  Description of Artificial Sequence: Synthetic peptide
RPKPDDLDI

SEQ ID NO: 43 - protein, 20 aa, Artificial Sequence
  Description of Artificial Sequence: Synthetic peptide
GRKKRRQRPPQRPRPDDLEI

SEQ ID NO: 44 - protein, 25 aa, Artificial Sequence
  Description of Artificial Sequence: Synthetic peptide
RQIKIWFQNRRMKWKKRPRPDDLEI

SEQ ID NO: 45 - protein, 25 aa, Artificial Sequence
  Description of Artificial Sequence: Synthetic peptide
RQIAIWFQNRRMKWAARPRPDDLEI

SEQ ID NO: 46 - protein, 18 aa, Artificial Sequence
  Description of Artificial Sequence: Synthetic peptide
RKKRRQRRRRPRPDDLEI

SEQ ID NO: 47 - protein, 30 aa, Artificial Sequence
  Description of Artificial Sequence: Synthetic polypeptide
TRSSRAGLQFPVGRVHRLLRKRPRPDDLEI

SEQ ID NO: 48 - protein, 35 aa, Artificial Sequence
  Description of Artificial Sequence: Synthetic polypeptide
GWTLNSAGYLLGKINKALAALAKKILRPRPDDLEI

SEQ ID NO: 49 - protein, 27 aa, Artificial Sequence
  Description of Artificial Sequence: Synthetic peptide
KLALKLALKALKAALKLARPRPDDLEI

SEQ ID NO: 50 - protein, 25 aa, Artificial Sequence
  Description of Artificial Sequence: Synthetic peptide
AAVALLPAVLLALLAPRPRPDDLEI

SEQ ID NO: 51 - protein, 19 aa, Artificial Sequence
  Description of Artificial Sequence: Synthetic peptide
VPMLKPMLKERPRPDDLEI

SEQ ID NO: 52 - protein, 37 aa, Artificial Sequence
  Description of Artificial Sequence: Synthetic polypeptide
MANLGYWLLALFVTMWTDVGLCKKRPKPRPRPDDLEI

SEQ ID NO: 53 - protein, 27 aa, Artificial Sequence
  Description of Artificial Sequence: Synthetic peptide
LLIILRRRIRKQAHAHSKRPRPDDLEI

SEQ ID NO: 54 - protein, 30 aa, Artificial Sequence
  Description of Artificial Sequence: Synthetic polypeptide
KETWWETWWTEWSQPKKKRKVRPRPDDLEI

SEQ ID NO: 55 - protein, 27 aa, Artificial Sequence
  Description of Artificial Sequence: Synthetic peptide
RGGRLSYSRRRFSTSTGRRPRPDDLEI

SEQ ID NO: 56 - protein, 24 aa, Artificial Sequence
  Description of Artificial Sequence: Synthetic peptide
SDLWEMMMVSLACQYRPRPDDLEI

SEQ ID NO: 57 - protein, 21 aa, Artificial Sequence
  Description of Artificial Sequence: Synthetic peptide
TSPLNIHNGQKLRPRPDDLEI

SEQ ID NO: 58 - skipped

SEQ ID NO: 59 - protein, 11 aa, Artificial Sequence
  Description of Artificial Sequence: Synthetic peptide
SRPTEKTIFII

SEQ ID NO: 60 - protein, 13 aa, Artificial Sequence
  Description of Artificial Sequence: Synthetic peptide
VCYDKSFPISHVR

SEQ ID NO: 61 - protein, 10 aa, Artificial Sequence
  Description of Artificial Sequence: Synthetic peptide
CFLSRPTEKT

SEQ ID NO: 62 - protein, 8 aa, Artificial Sequence
  Description of Artificial Sequence: Synthetic peptide
LSRPTEKT

SEQ ID NO: 63 - protein, 7 aa, Artificial Sequence
  Description of Artificial Sequence: Synthetic peptide
SRPTEKT

SEQ ID NO: 64 - protein, 10 aa, Artificial Sequence
  Description of Artificial Sequence: Synthetic peptide
VDCFLSRPTE

SEQ ID NO: 65 - protein, 8 aa, Artificial Sequence
  Description of Artificial Sequence: Synthetic peptide
VDCFLSRP

SEQ ID NO: 66 - protein, 6 aa, Artificial Sequence
  Description of Artificial Sequence: Synthetic peptide
VDCFLS

SEQ ID NO: 67 - protein, 23 aa, Artificial Sequence
  Description of Artificial Sequence: Synthetic peptide
GRKKRRQRPPQVDCFLSRPTEKT

SEQ ID NO: 68 - protein, 28 aa, Artificial Sequence
  Description of Artificial Sequence: Synthetic peptide
RQIKIWFQNRRMKWKKVDCFLSRPTEKT

SEQ ID NO: 69 - protein, 28 aa, Artificial Sequence
  Description of Artificial Sequence: Synthetic peptide
RQIAIWFQNRRMKWAAVDCFLSRPTEKT

SEQ ID NO: 70 - protein, 21 aa, Artificial Sequence
  Description of Artificial Sequence: Synthetic peptide
RKKRRQRRRVDCFLSRPTEKT

SEQ ID NO: 71 - protein, 33 aa, Artificial Sequence
  Description of Artificial Sequence: Synthetic polypeptide
TRSSRAGLQFPVGRVHRLLRKVDCFLSRPTEKT

SEQ ID NO: 72 - protein, 38 aa, Artificial Sequence
  Description of Artificial Sequence: Synthetic polypeptide
GWTLNSAGYLLGKINKALAALAKKILVDCFLSRPTEKT

SEQ ID NO: 73 - protein, 30 aa, Artificial Sequence
  Description of Artificial Sequence: Synthetic polypeptide
KLALKLALKALKAALKLAVDCFLSRPTEKT

SEQ ID NO: 74 - protein, 28 aa, Artificial Sequence
  Description of Artificial Sequence: Synthetic peptide
AAVALLPAVLLALLAPVDCFLSRPTEKT

SEQ ID NO: 75 - protein, 22 aa, Artificial Sequence
  Description of Artificial Sequence: Synthetic peptide
VPMLKPMLKEVDCFLSRPTEKT

SEQ ID NO: 76 - protein, 40 aa, Artificial Sequence
  Description of Artificial Sequence: Synthetic polypeptide
MANLGYWLLALFVTMWTDVGLCKKRPKPVDCFLSRPTEKT

SEQ ID NO: 77 - protein, 30 aa, Artificial Sequence
  Description of Artificial Sequence: Synthetic polypeptide
LLIILRRRIRKQAHAHSKVDCFLSRPTEKT

SEQ ID NO: 78 - protein, 33 aa, Artificial Sequence
  Description of Artificial Sequence: Synthetic polypeptide
KETWWETWWTEWSQPKKKRKVVDCFLSRPTEKT

SEQ ID NO: 79 - protein, 30 aa, Artificial Sequence
  Description of Artificial Sequence: Synthetic polypeptide
RGGRLSYSRRRFSTSTGRVDCFLSRPTEKT

SEQ ID NO: 80 - protein, 27 aa, Artificial Sequence
  Description of Artificial Sequence: Synthetic peptide
SDLWEMMMVSLACQYVDCFLSRPTEKT

SEQ ID NO: 81 - protein, 24 aa, Artificial Sequence
  Description of Artificial Sequence: Synthetic peptide
TSPLNIHNGQKLVDCFLSRPTEKT

SEQ ID NO: 82 - protein, 11 aa, Artificial Sequence
  Description of Artificial Sequence: Synthetic peptide
FEVAFLLIQWI

SEQ ID NO: 83 - protein, 11 aa, Artificial Sequence
  Description of Artificial Sequence: Synthetic peptide
LLIQWYIGFSL

SEQ ID NO: 84 - protein, 16 aa, Artificial Sequence
  Description of Artificial Sequence: Synthetic peptide
SLSAVYTCKRDPCPHQ

SEQ ID NO: 85 - protein, 11 aa, Artificial Sequence
  Description of Artificial Sequence: Synthetic peptide
SRPTEKTIFII

SEQ ID NO: 86 - protein, 13 aa, Artificial Sequence
  Description of Artificial Sequence: Synthetic peptide
LGTAVESAWGDEQ

SEQ ID NO: 87 - protein, 12 aa, Artificial Sequence
  Description of Artificial Sequence: Synthetic peptide
QSAFRCNTQQPG

SEQ ID NO: 88 - protein, 12 aa, Artificial Sequence
  Description of Artificial Sequence: Synthetic peptide
QQPGCENVCYDK

SEQ ID NO: 89 - protein, 13 aa, Artificial Sequence
  Description of Artificial Sequence: Synthetic peptide
VCYDKSFPISHVR

SEQ ID NO: 90 - protein, 18 aa, Artificial Sequence
  Description of Artificial Sequence: Synthetic peptide
KRDPCHQVDCFLSRPTEK

SEQ ID NO: 91 - protein, 6 aa, Artificial Sequence
  Description of Artificial Sequence: Synthetic peptide
  MOD_RES (1)..(1): D-Tyr
  MOD_RES (3)..(3): D-4Hyp
  MOD_RES (5)..(5): D-Ala
YPXGAG

SEQ ID NO: 92 - protein, 28 aa, Artificial Sequence
  Description of Artificial Sequence: Synthetic peptide
RVIRVWFQNKRCKDKKVDCFLSRPTEKT

SEQ ID NO: 93 - protein, 39 aa, Artificial Sequence
  Description of Artificial Sequence: Synthetic polypeptide
GALFLGFLGAAGSTMGAWSQPKKKRKVVDCFLSRPTEKT

SEQ ID NO: 94 - protein, 39 aa, Artificial Sequence
  Description of Artificial Sequence: Synthetic polypeptide
GALFLAFLAAALSLMGLWSQPKKKRRVVDCFLSRPTEKT

SEQ ID NO: 95 - protein, 39 aa, Artificial Sequence
  Description of Artificial Sequence: Synthetic polypeptide
MYVFYVMYDGFSMQRLVKCNAWPCPNTVDCFVSRPTEKT

SEQ ID NO: 96 - protein, 39 aa, Artificial Sequence
  Description of Artificial Sequence: Synthetic polypeptide
MYVFYFLYNGYHLPWVLKCGIDPCPNLVDCFISRPTEKT

SEQ ID NO: 97 - protein, 39 aa, Artificial Sequence
  Description of Artificial Sequence: Synthetic polypeptide
LYIFHRLYKDYDMPRVVACSVEPCPHTVDCYISRPTEKK

SEQ ID NO: 98 - protein, 40 aa, Artificial Sequence
  Description of Artificial Sequence: Synthetic polypeptide
LYLLHTLWHGFNMPRLVQCANVAPCPNIVDCYIARPTEKK

SEQ ID NO: 99 - protein, 39 aa, Artificial Sequence
  Description of Artificial Sequence: Synthetic polypeptide
LYVFHSFYPKYILPPVVKCHADPCPNIVDCFISKPSEKN

SEQ ID NO: 100 - protein, 39 aa, Artificial Sequence
  Description of Artificial Sequence: Synthetic polypeptide
MYVFYLLYPGYAMVRLVKCDVYPCPNTVDCFVSRPTEKT

SEQ ID NO: 101 - protein, 32 aa, Artificial Sequence
  Description of Artificial Sequence: Synthetic polypeptide
LYGWTMEPVFVCQRAPCPYLVDCFVSRPTEKT

SEQ ID NO: 102 - protein, 32 aa, Artificial Sequence; identical to SEQ ID NO: 101
  Description of Artificial Sequence: Synthetic polypeptide

SEQ ID NO: 103 - protein, 38 aa, Artificial Sequence
  Description of Artificial Sequence: Synthetic polypeptide
GALHYFLFGFLAPKKFPCTRPPCTGVVDCYVSRPTEKS

SEQ ID NO: 104 - protein, 38 aa, Artificial Sequence; identical to SEQ ID NO: 12
  Description of Artificial Sequence: Synthetic polypeptide

SEQ ID NO: 105 - protein, 38 aa, Artificial Sequence
  Description of Artificial Sequence: Synthetic polypeptide
IAGQYFLYGFELKPLYRCDRWPCPNTVDCFISRPTEKT

SEQ ID NO: 106 - protein, 38 aa, Artificial Sequence
  Description of Artificial Sequence: Synthetic polypeptide
LVGQYLLYGFEVRPFFPCSRQPCPHVVDCFVSRPTEKT

SEQ ID NO: 107 - protein, 38 aa, Artificial Sequence
  Description of Artificial Sequence: Synthetic polypeptide
IVGQYFIYGIFLTTLHVCRRSPCPHPVNCYVSRPTEKN

SEQ ID NO: 108 - protein, 46 aa, Artificial Sequence
  Description of Artificial Sequence: Synthetic polypeptide
LTAVGGESIYYDEQSKFVCNTEQPGCENVCYDAFAPLSHVRFWVFQ

SEQ ID NO: 109 - protein, 15 aa, Artificial Sequence
  Description of Artificial Sequence: Synthetic peptide
LTAVGGESIYYDEQS

SEQ ID NO: 110 - protein, 13 aa, Artificial Sequence
  Description of Artificial Sequence: Synthetic peptide
DEQSKFVCNTEQP

SEQ ID NO: 111 - protein, 13 aa, Artificial Sequence
  Description of Artificial Sequence: Synthetic peptide
TEQPGCENVCYDA

SEQ ID NO: 112 - protein, 13 aa, Artificial Sequence
  Description of Artificial Sequence: Synthetic peptide
VCYDAFAPLSHVR

SEQ ID NO: 113 - protein, 12 aa, Artificial Sequence
  Description of Artificial Sequence: Synthetic peptide
APLSHVRFWVFQ

SEQ ID NO: 114 - protein, 47 aa, Artificial Sequence
  Description of Artificial Sequence: Synthetic polypeptide
FEVGFLIGQYFLYGFQVHPFYVCSRLPCHPKIDCFISRPTEKTIFLL

SEQ ID NO: 115 - protein, 11 aa, Artificial Sequence
  Description of Artificial Sequence: Synthetic peptide
FEVGFLIGQYF

SEQ ID NO: 116 - protein, 12 aa, Artificial Sequence
  Description of Artificial Sequence: Synthetic peptide
LIGQYFLYGFQV

SEQ ID NO: 117 - protein, 14 aa, Artificial Sequence
  Description of Artificial Sequence: Synthetic peptide
GFQVHPFYVCSRLP

SEQ ID NO: 118 - protein, 12 aa, Artificial Sequence
  Description of Artificial Sequence: Synthetic peptide
SRLPCHPKIDCF

SEQ ID NO: 119 - protein, 12 aa, Artificial Sequence
  Description of Artificial Sequence: Synthetic peptide
IDCFISRPTEKT

SEQ ID NO: 120 - protein, 11 aa, Artificial Sequence
  Description of Artificial Sequence: Synthetic peptide
SRPTEKTIFLL

SEQ ID NO: 121 - protein, 11 aa, Artificial Sequence
  Description of Artificial Sequence: Synthetic peptide
SRPTEKTIFII

SEQ ID NO: 122 - protein, 10 aa, Artificial Sequence
  Description of Artificial Sequence: Synthetic peptide
YVCSRLPCHP

SEQ ID NO: 123 - protein, 11 aa, Artificial Sequence
  Description of Artificial Sequence: Synthetic peptide
QVHPFYVCSRL

SEQ ID NO: 124 - protein, 13 aa, Artificial Sequence
  Description of Artificial Sequence: Synthetic peptide
FEVGFLIGQYFLY

SEQ ID NO: 125 - protein, 12 aa, Artificial Sequence
  Description of Artificial Sequence: Synthetic peptide
GQYFLYGFQVHP

SEQ ID NO: 126 - protein, 12 aa, Artificial Sequence
  Description of Artificial Sequence: Synthetic peptide
GFQVHPFYVCSR

SEQ ID NO: 127 - protein, 12 aa, Artificial Sequence
  Description of Artificial Sequence: Synthetic peptide
AVGGESIYYDEQ

SEQ ID NO: 128 - protein, 12 aa, Artificial Sequence
  Description of Artificial Sequence: Synthetic peptide
YDEQSKFVCNTE

SEQ ID NO: 129 - protein, 12 aa, Artificial Sequence
  Description of Artificial Sequence: Synthetic peptide
NTEQPGCENVCY

SEQ ID NO: 130 - protein, 12 aa, Artificial Sequence
  Description of Artificial Sequence: Synthetic peptide
CYDAFAPLSHVR

SEQ ID NO: 131 - protein, 12 aa, Artificial Sequence
  Description of Artificial Sequence: Synthetic peptide
FAPLSHVRFWVF

SEQ ID NO: 132 - protein, 5 aa, Artificial Sequence
  Description of Artificial Sequence: Synthetic peptide
LIGQY

SEQ ID NO: 133 - protein, 5 aa, Artificial Sequence
  Description of Artificial Sequence: Synthetic peptide
QVHPF

SEQ ID NO: 134 - protein, 5 aa, Artificial Sequence
  Description of Artificial Sequence: Synthetic peptide
YVCSR

SEQ ID NO: 135 - protein, 5 aa, Artificial Sequence
  Description of Artificial Sequence: Synthetic peptide
SRLPC

SEQ ID NO: 136 - protein, 5 aa, Artificial Sequence
  Description of Artificial Sequence: Synthetic peptide
LPCHP

SEQ ID NO: 137 - protein, 5 aa, Artificial Sequence
  Description of Artificial Sequence: Synthetic peptide
GESIY

SEQ ID NO: 138 - protein, 6 aa, Artificial Sequence
  Description of Artificial Sequence: Synthetic peptide
YDEQSK

SEQ ID NO: 139 - protein, 6 aa, Artificial Sequence
  Description of Artificial Sequence: Synthetic peptide
SKFVCN

SEQ ID NO: 140 - protein, 8 aa, Artificial Sequence
  Description of Artificial Sequence: Synthetic peptide
TEQPGCEN

SEQ ID NO: 141 - protein, 8 aa, Artificial Sequence
  Description of Artificial Sequence: Synthetic peptide
VCYDAFAP

SEQ ID NO: 142 - protein, 10 aa, Artificial Sequence
  Description of Artificial Sequence: Synthetic peptide
LSHVRFWVFQ

SEQ ID NO: 143 - protein, 14 aa, Artificial Sequence
  Description of Artificial Sequence: Synthetic peptide
LIQYFLYGFQVHPF

SEQ ID NO: 144 - protein, 13 aa, Artificial Sequence
  Description of Artificial Sequence: Synthetic peptide
VHPFYCSRLPCHP

SEQ ID NO: 145 - protein, 22 aa, Artificial Sequence
  Description of Artificial Sequence: Synthetic peptide
VGGESIYYDEQSKFVCNTEQPG

SEQ ID NO: 146 - protein, 22 aa, Artificial Sequence
  Description of Artificial Sequence: Synthetic peptide
TEQPGCENVCYDAFAPLSHVRF

SEQ ID NO: 147 - protein, 14 aa, Artificial Sequence
  Description of Artificial Sequence: Synthetic peptide
AFAPLSHVRFWVFQ

SEQ ID NO: 148 - protein, 16 aa, Artificial Sequence
  Description of Artificial Sequence: Synthetic peptide
IDCFISRPTEKTIFLL

SEQ ID NO: 149 - protein, 11 aa, Artificial Sequence
  Description of Artificial Sequence: Synthetic peptide
DCFISRPTEKT

SEQ ID NO: 150 - protein, 7 aa, Artificial Sequence
  Description of Artificial Sequence: Synthetic peptide
SRPTEKT

SEQ ID NO: 151 - protein, 38 aa, Artificial Sequence; identical to SEQ ID NO: 13
  Description of Artificial Sequence: Synthetic polypeptide

SEQ ID NO: 152 - protein, 16 aa, Unknown
  Description of Unknown: ANTP peptide
RQPKIWFPNRRKPWKK

SEQ ID NO: 153 - protein, 11 aa, Human immunodeficiency virus
GRKKRRQRPPQ

SEQ ID NO: 154 - protein, 26 aa, Unknown
  Description of Unknown: Transportan peptide
GWTLNSAGYLLGKINKALAALAKKIL

SEQ ID NO: 155 - protein, 21 aa, Unknown
  Description of Unknown: Buforin II peptide
TRSSRAGLQFPVGRVHRLLRK

SEQ ID NO: 156 - protein, 9 aa, Unknown
  Description of Unknown: Tat peptide
RKKRRQRRR

SEQ ID NO: 157 - protein, 16 aa, Drosophila sp.
RQIKIWFQNRRMKWKK

SEQ ID NO: 158 - protein, 18 aa, Unknown
  Description of Unknown: MAP peptide
KLALKLALKALKAALKLA

SEQ ID NO: 159 - protein, 16 aa, Unknown
  Description of Unknown: K-FGF peptide
AAVALLPAVLLALLAP

SEQ ID NO: 160 - protein, 10 aa, Unknown
  Description of Unknown: Ku70 peptide
VPMLKPMLKE

SEQ ID NO: 161 - protein, 28 aa, Unknown
  Description of Unknown: Prion peptide
MANLGYWLLALFVTMWTDVGLCKKRPKP

SEQ ID NO: 162 - protein, 18 aa, Unknown
  Description of Unknown: pVEC peptide
LLIILRRRIRKQAHAHSK

SEQ ID NO: 163 - protein, 21 aa, Unknown
  Description of Unknown: Pep-1 peptide
KETWWETWWTEWSQPKKKRRV

SEQ ID NO: 164 - protein, 18 aa, Unknown
  Description of Unknown: SynB1 peptide
RGGRLSYSRRRFSTSTGR

SEQ ID NO: 165 - protein, 15 aa, Unknown
  Description of Unknown: Pep-7 peptide
SDLWEMMMVSLACQY

SEQ ID NO: 166 - protein, 12 aa, Unknown
  Description of Unknown: HN-1 peptide
TSPLNIHNGQKL

SEQ ID NO: 167 - protein, 16 aa, Unknown
  Description of Unknown: plsl peptide
RVIRVWFQNKRCKDKK

SEQ ID NO: 168 - protein, 27 aa, Unknown
  Description of Unknown: MGB Peptide P-beta sequence
GALFLGFLGAAGSTMGAWSQPKKKRKV

SEQ ID NO: 169 - protein, 27 aa, Unknown
  Description of Unknown: MGB Peptide P-alpha sequence
GALFLAFLAAALSLMGLWSQPKKKRRV

SEQ ID NO: 170 - protein, 7 aa, Hepatitis B virus
LCLRPVG

SEQ ID NO: 171 - protein, 14 aa, Artificial Sequence
  Description of Artificial Sequence: Synthetic peptide
  MOD_RES (1)..(2): Any amino acid with C12 alkyl side chain
  See specification as filed for detailed description of substitutions and preferred embodiments
XXVDCFLSRPTEKT

SEQ ID NO: 172 - protein, 4 aa, Artificial Sequence
  Description of Artificial Sequence: Synthetic peptide
SHVR

SEQ ID NO: 173 - protein, 6 aa, Artificial Sequence
  Description of Artificial Sequence: Synthetic peptide
SRPTEK

SEQ ID NO: 174 - protein, 8 aa, Artificial Sequence
  Description of Artificial Sequence: Synthetic peptide
RRNYRRNY

SEQ ID NO: 175 - protein, 14 aa, Artificial Sequence
  Description of Artificial Sequence: Synthetic peptide
  MOD_RES (1)..(2): Any amino acid with a hydrogen or alkyl side chain
  See specification as filed for detailed description of substitutions and preferred embodiments
XXVDCFLSRPTEKT